(19) United States
(12) Patent Application Publication
(10) Pub. No.: US 2008/0244765 A1
(43) Pub. Date: Oct. 2, 2008

(54) METHODS AND COMPOSITIONS FOR POLLINATION DISRUPTION

(75) Inventors: Zuo-Yu Zhao, Johnston, IA (US); Youngzhong Wu, Johnston, IA (US); Michael Lassner, Urbandale, IA (US); Howard Hershey, Cumming, IA (US)

Correspondence Address: PAT SWEENEY;ATTN: PHI, 1835 PLEASANT ST., WEST DES MOINES, IA, 50265, US

(73) Assignee: Pioneer Hi-Bred International, Inc., Johnston, IA (US)

(21) Appl. No.: 12/062,212

(22) Filed: Apr. 3, 2008

Related U.S. Application Data
(63) Continuation-in-part of application No. 11/471,202, filed on Jun. 20, 2006.
Application No. 11/471,202 is a continuation-in-part of application No. 11/014,071, filed on Dec. 16, 2004.

(51) Int. Cl. C12N 15/11; A01H 5/00; C12N 5/10; C12N 1/19; C12N 1/21; C12N 15/63; A01H 1/02; B07C 5/342

(52) U.S. Cl. 800/260; 536/24.1; 800/298; 800/320.1; 800/320; 435/419; 435/254.2; 435/252.3; 800/319; 435/320.1; 800/278; 209/509

(57) ABSTRACT

Pollination process is disrupted by use of a pollination-disruption polynucleotide that renders the pollen unable to fertilize a sexually compatible ovule. The non-lethal nature of the pollen disruption polynucleotide is advantageous, particularly when operably linked to a transgenic polynucleotide of interest and prevents transmission of the polynucleotide through pollen. non-lethal markers are employed in an embodiment in which transgenic and non-transgenic seed can be sorted. A recombinase excision system is employed in an embodiment to activate the pollen disruption polynucleotide.

This application is a continuation-in-part of previously filed and co-pending applications U.S. Ser. No. 11/471,202 filed Jun. 20, 2006 and U.S. Ser. No. 11/014,071, filed Dec. 16, 2004, both of which are incorporated herein in their entirety by reference.

### BACKGROUND OF THE INVENTION

Transgenic crops and the application of biotechnology are dramatically altering seed and agrochemical businesses throughout the world. The seeds of commercially important crops have been genetically engineered to be resistant to herbicides and pests, especially insect pests. The uncontrolled transmission of heterologous traits in commercially important crop plants is currently a major concern throughout the world and especially within the agricultural community.

The biotechnology industry is interested in transferring traits such as tolerances to drought, insects, diseases, salinity, frost and herbicides into cultivated plants which might confer an adaptive advantage over wild plants. Interest has increased in preventing the transmission of heterologous traits from genetically modified organisms.

The biotechnology industry is also interested in propagating important recessive agronomic traits such as recessive male sterile mutation for hybrid seed production. Several naturally-occurring systems including self-incompatibility and cytoplasmic genetic male sterility (CMS) have been exploited for pollination control, each with its own disadvantages and advantages. Genetically engineered male sterility systems have also been reported, but the lack of an efficient method to propagate the male sterile plants limits their use for commercial hybrid seed production (E. Perez-Prat, M. M. van Lookeren Campagne, Trends Plant Sci., 7, 199-203, 2002.). Thus, there is a need to develop an efficient pollination control system for commercial hybrid seeds production. For these and other reasons, there is a need for the present invention. All references cited herein are incorporated herein by reference.

### BRIEF SUMMARY OF THE INVENTION

Methods and constructs to make transgenic pollen malfunctional and to identify transgenic seeds by color sorting are shown. An embodiment provides that a non-lethal pollination-disruption polynucleotide is linked with a promoter directing expression to pollen, the expression of which renders the transgenic pollen grains unable to fertilize a sexually compatible ovule. The polynucleotide can be linked to a transgenic polynucleotide of interest, thereby significantly blocking transmission through pollen of the transgene. In an embodiment, non-lethal markers are used to select seeds having the transgene and the pollination-disruption polynucleotide, and in a preferred embodiment, sorting of seed may occur by color sorting. The use of color sorting, in combination with the non-lethal pollination-disruption polynucleotide can reduce the probability of a transgenic seed released into the environment. Yet another embodiment provides for activation of the pollination-disruption polynucleotide by a recombinase excision system. Use of such systems for turning the system on are also disclosed.

### DETAILED DESCRIPTION OF THE PREFERRED EMBODIMENT

The present invention in one aspect relates to methods of blocking or reducing genetically modified organisms (GMO) pollen flow using a “non-lethal” approach. In this aspect, at least one transgenic polynucleotide of interest is linked to a pollination-disruption and color sorting construct as described hererin. The pollination-disruption and color sorting construct contains a pollination-disruption polynucleotide that makes transgenic pollen malfunctional and thus prevents the transgenic pollen grains from achieving fertilization. The system is highly effective and as little as 0.01% transgenic pollen grains are expected to function normally. Any transgenic seeds derived from the pollination by such functional transgenic pollen, as discussed below, can be further sorted and separated, a process that in a preferred embodiment is particularly efficient when using color marking of the transgenic seed. This allows use of such systems as commercial High-Speed Color Sorters. The pollination-disruption and color sorting constructs and methods described herein can be used directly to produce traditional (non-transgenic) hybrid seeds as well as transgenic GMO hybrid seeds using various breeding methods, such as a CMS system. In another aspect, the constructs may be used to propagate recessive agronomic traits such as recessive male sterile mutations for hybrid seed production.

Sorting of transgenic seed from those not containing the construct is aided in one embodiment by use of non-lethal markers, that is markers which do not require destruction of the plant tissue. Visible selection markers are particularly useful, and in an embodiment are those markers which provide a color to the plant tissue when present. In an embodiment, nucleotide sequences encoding beta carotene are useful for selection, as it provides a golden color to tissue. When operably linked with a promoter directing expression to seed tissue, one can employ visual seed sorting by color to distinguish seed having the construct from that which does not contain the construct.

Yet further embodiments provide for use of a recombinase excision system, such as FLP/frt and CRE/lox, in a system which activates the pollination disruption polynucleotide. When combined with two color markers for distinguishing tissue where excision has occurred from that where it has not, the selection process is further aided.

Plants suitable for purposes of the methods disclosed herein can be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, switchgrass, rapeseed, clover, tobacco, turfgrass, carrot, cotton, alfalfa, rice, potato, eggplant, cucumber, Arabidopsis thaliana, and woody plants such as coniferous and deciduous trees. Thus, a transgenic plant or genetically modified plant cell of the invention can be an angiosperm or gymnosperm. The methods of the invention are particularly useful when applied to use in crops of corn, sorghum, rice, and other grasses. Sorghum, for example, presents a particular challenge when attempting to produce a male sterile line to cross with a female line. Due to the physical structure of the plant it is not possible to detassle sorghum. Far less is known about sorghum transformation, and the cytoplasmic male sterility system is typically employed instead, which presents issues of concern about side effects of such a system. However, the inventors have found that one can achieve high male sterility rates in sorghum using the processes of the invention, while preventing transgene transmission through pollen.

### DEFINITIONS

As used herein, the term “allele” refers to any of several alternative forms of a gene.

As used herein, the term “crop plant” refers to any plant grown for any commercial purpose, including, but not limited to the following purposes: seed production, hay production, ornamental use, fruit production, berry production, vegetable production, oil production, protein production, forage production, animal grazing, golf courses, lawns, flower production, landscaping, erosion control, green manure, improving soil tilth/health, producing pharmaceutical products/drugs, producing food additives, smoking products, pulp production and wood production.

As used herein, the term “cross pollination” or “cross-fertilizing” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

The term “female” refers to a plant that produces ovules. Female plants generally produce seeds after fertilization. A plant designated as a “female plant” may contain both male and female sexual organs. Alternatively, the “female plant” may only contain female sexual organs either naturally (e.g., in dioecious species) or due to emasculation (e.g., by detasselling) or from male-sterility.

As used herein, the term “filial generation” refers to any of the generations of cells, tissues or organisms following a particular parental generation. The generation resulting from a mating of the parents is the first filial generation (designated as “F1” or “F₁”), while that resulting from crossing of F1 individuals is the second filial generation (designated as “F2” or “F₂”).

The term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “hemizygous” refers to a cell, tissue or organism in which a gene is present single dose in a genotype, as a gene in a haploid cell or organism, a sex-linked gene in the heterogametic sex, or a gene in a segment of chromosome in a diploid cell or organism where its partner segment has been deleted. It also includes the situation of an absence of an allele, as when a transgenic construct is introduced into the plant, and thus is present single dose in the genotype.

A “heterologous polynucleotide” or a “heterologous nucleic acid” or an “exogenous DNA segment” refers to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “homozygous” refers to the presence of identical alleles at one or more loci in homologous chromosomal segments. Heterozygous is where there are two different alleles at the same locus.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T₀) plant regenerated from material of that line; (b) has a pedigree comprised of a T₀ plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by different sequences.

The term “male” refers to a plant that produces pollen grains. The “male plant” generally refers to the sex that produces male gametes for fertilizing ova. A plant designated as a “male plant” may contain both male and female sexual organs. Alternatively, the “male plant” may only contain male sexual organs either naturally (e.g., in dioecious species) or due to emasculation (e.g., by removing the ovary).

As used herein, the term “mass selection” refers to a form of selection in which individual plants are selected and the next generation propagated from the aggregate of their seeds.

As used herein, the terms “nucleic acid” or “polynucleotide” refer to deoxyribonucleotides or ribonucleotides and polymers thereof in either single- or double-stranded form. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

As used herein, a nucleotide segment is referred to as “operably linked” when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof. The expression cassette can include one or more enhancers in addition to the promoter. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements. An example of one such enhancer is the 35S enhancer, which can be a single enhancer, or duplicated. See for example, McPherson et al, U.S. Pat. No. 5,322,938.

As used herein, the term “promoter directing expression to pollen” or “pollen-specific promoter” refers to a nucleic acid sequence that regulates the expression of nucleic acid sequences selectively in the cells or tissues of a plant essential to pollen formation and/or function and/or limits the expression of a nucleic acid sequence to the period of pollen formation in the plant. It may express at higher levels in the pollen tissue compared to other plant tissue, may express highly in the pollen, express more in the pollen tissue than in other plant tissue, or express exclusively in the pollen tissue.

The term “recombinant” is used herein to refer to a nucleic acid molecule that is manipulated outside of a cell, including two or more linked heterologous nucleotide sequences.

The term “plant” is used broadly herein to include any plant at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In another aspect, the present invention provides regenerable cells for use in tissue culture or inbred corn plant W16090. The tissue culture will preferably be capable of regenerating plants having the physiological and morphological characteristics of the foregoing inbred corn plant, and of regenerating plants having substantially the same genotype as the foregoing inbred corn plant. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, the present invention provides corn plants regenerated from the tissue cultures of the invention.

As used herein, the term “self pollinated” or “self-pollination” means the pollen of one flower on one plant is applied (artificially or naturally) to the ovule (stigma) of the same or a different flower on the same plant.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “introduction” refers to introducing into plant cells, cell cultures, organisms, plants, and progeny of plants which have received a foreign or modified gene by one of the various methods of transformation or breeding from a transgenic plant, as discussed below, wherein the foreign or modified gene is from the same or different species than the species of the plant, or organism, receiving the foreign or modified gene introducing the gene directly or through transformation and subsequent breeding the transgenic gene.

As used herein, the term “variety” refers to a subdivision of a species, consisting of a group of individuals within the species that are distinct in form or function from other similar arrays of individuals.

As used herein, the term “vector” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). Thus, the maize preferred codon for a particular amino acid may be derived from known gene sequences from maize. Maize codon usage for 28 genes from maize plants are listed in Table 4 of Murray et al., supra.

With reference to nucleic acid molecules, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize or sorghum host cell.

The term “hybridization complex” includes reference to a duplex nucleic acid structure formed by two single-stranded nucleic acid sequences selectively hybridized with each other.

The term “introduced” in the context of inserting a nucleic acid into a cell, includes “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). When referring to “introduction” of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. For a discussion of plant breeding techniques, see Poehlman (1995) Breeding Field Crops. AVI Publication Co., Westport Conn., 4^{th} Edit. Backcrossing methods may be used to introduce a gene into the plants. This technique has been used for decades to introduce traits into a plant. An example of a description of this and other plant breeding methodologies that are well known can be found in references such as Poelman, supra, and Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and may be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 0.11% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

Nucleic acid sequences and amino acid sequences can be compared using computer programs that align the similar sequences of the nucleic or amino acids thus define the differences. The BLAST programs (NCBI) and parameters used therein are used by many practitioners to align amino acid sequence fragments. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by Best-Fit program may also be used to compare sequence identity and similarity.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” refers to a coding sequence or sequences operably linked to appropriate regulatory sequences and inserted into a vector for transforming a cell. Such a nucleic acid construct may contain a coding sequence for a gene product of interest, along with a marker gene and/or a reporter gene.

The term “marker gene” refers to a gene encoding a product that, when expressed, confers a phenotype or genotype on a transformed cell providing identification of cells expressing the marker.

As used herein, the term “malfunctional” pollen refers to pollen that may be viable within anthers but cannot pollinate or fertilize a sexually compatible plant. For example, malfunctional pollen may be incapable of fertilization for a number of reasons including the inability of the pollen to re-hydrate, germinate, produce a pollen tube, or release a sperm cell, thereby preventing fertilization of a sexually compatible ovule.

The inventors have discovered compositions and methods for producing plants that produce malfunctional pollen so that the transmission of a transgenic polynucleotide from genetically modified plants is blocked. Malfunctional pollen may be achieved using a pollen-specific promoter operably linked to a pollination-disruption polynucleotide that renders the pollen malfunctional. In one aspect, a recombinant nucleotide construct includes the pollen-specific promoter operably linked to a pollination-disruption polynucleotide and a transgenic polynucleotide of interest, herein after, referred to as the pollination-disruption construct.

Pollen-specific promoters include those promoters active during pollen development, as well as those promoters active during pollen germination or active in anther and/or pollen or in tissues that give rise to anther cells and/or pollen or pollen compartments, including but not limited to the amyloplastid, mitochondria, protein bodies, oil bodies or other compartments in pollen, including those that store energy sources and enzymes. Suitable pollen-specific promoters drive expression specifically, preferentially in pollen and may be expressed in other parts of the plant as well. Pollen specific promoters include, for example, an MS45 gene promoter (U.S. Pat. No. 6,037,523), a 5126 gene promoter (U.S. Pat. No. 5,837,851), a BS7 gene promoter (WO 02/063021), an SB200 gene promoter, (WO 02/26789) a TA29 gene promoter (Nature 347:737 (1990)), a PG47 gene promoter (U.S. Pat. No. 5,412,085; U.S. Pat. No. 5,545,546; Plant J 3(2):261-271 (1993)), P67 or P95 (See US publication 20050246796) promoters, an SGB6 gene promoter (U.S. Pat. No. 5,470,359) a G9 gene promoter (U.S. Pat. Nos. 5,837,850; 5,589,610), or the like. Additional tissue-specific or stage-specific regulatory elements include the Zn13 promoter, which is a pollen-specific promoter (Hamilton et al., Plant Mol. Biol. 18:211-218, 1992); and sperm cell-specific promoters.

Pollen-specific promoters have been identified in many plant species such as maize, rice, tomato, tobacco, Arabidopsis, Brassica, and others (Odell, T. O., et al. (1985) Nature 313:810-812; Marrs, K. A., et al, (1993) Dev Genet, Vol. 14/1:27-41; Kim, (1992) Transgenic Res, Vol. 1/4:188-94; Carpenter, J. L., et al. (1992) Plant Cell Vol. 4/5:557-71; Albani, D. et al., (1992) Plant J. 2/3:331-42; Rommens, C. M., et al. (1992), Mol. Gen. Genet., Vol. 231/3:433-41; Kloeckener-Gruissem, et al., (1992) Embo J, Vol. 11/1:157-66; Hamilton, D. A. et al., (1992), Plant Mol Biol, Vol. 18/2:211-18; Kyozuka, J., et al. (1991), Mol. Gen. Genet., Vol. 228/1-2:40-8; Albani, D. et. al (1991) Plant Mol Biol Vol. 16/4:501-13; Twell, D. et al. (1991) Genes Dev. 5/3:496-507; Thorsness, M. K. et al., (1991) Dev. Biol Vol. 143/1:173-84; McCormick, S. et al. (1991) Symp Soc Exp Biol Vol. 45:229-44; Guerrero, F. D. et al. (1990) Mol Gen Genet Vol 224/2:161-8; Twell, D. et al., (1990) Development Vol. 109/3:705-13; Bichler, J. et al. (1990), Eur J Biochem Vol. 190/2:415-26; van Tunen, et al. (1990), Plant Cell Vol 2/5:393-401; Siebertz, B. et al., (1989) Plant Cell Vol 1/10:961-8; Sullivan, T. D. et al, (1989) Dev Genet Vol 10/6:412-24; Chen, J. et al. (1987), Genetics Vol 116/3:469-77). Several other examples of pollen-specific promoters can be found-in GenBank. Additional promoters are also provided in U.S. Pat. Nos. 5,086,169; 5,756,324; 5,633,438; 5,412,085; 5,545,546 and 6,172,279.

In one aspect, a suitable pollination-disruption polynucleotide encodes a protein involved in disrupting the pollination process.

Pollination process in higher plants involves many continuous steps including the development of pollen grains in the anthers, the release and landing of pollen on stigma, pollen-stigma cell recognition, pollen re-hydration, germination and pollen tube growth, the release of sperm cells and the union of sperm cells and egg cells (fertilization). Thus, the pollination-disruption polynucleotides may disrupt the pollination process at any number of stages, including those described above, to control genetic flow.

As used herein, the term “pollination-disruption polynucleotide” refers to any polynucleotide, including but not limited to cDNA, RNA, or genomic nucleic acid sequences that express a product that is not toxic to cells but makes the pollen malfunctional, for example, renders the pollen unrecognizable by stigma cells or incompetent to re-hydrate, germinate and produce pollen tube to fertilize a sexually compatible ovule. Thus, the pollination-disruption polynucleotide does not kill the pollen, a system used in other processes, such as that described in the patents WO 93/25695 and US20020144305A1 in which pollen-lethal genes or suicide genes, represented by the RNase gene, significantly disrupt the metabolism, functioning and/or development which cause pollen death during pollen development within anthers. In another aspect, the pollination-disruption polynucleotide is not toxic to plant cell. Non-cytotoxic gene here means that a gene, where overexpressed in a cell, does not disrupt the fundamental metabolism which is required for cell viability, and thus does not kill the cells. It interferes with a biochemical pathway that is not required for cell viability. These biochemical pathways include storage lipid and storage starch biosysnthesis processes, and biosyntheses of molecules for cell-cell (such as pollen-stigma) recognition. In avocado and olive, for example, the mesocarp lipids are not thought to contribute to the germination or growth of the seedling but are used to facilitate seed dispersal by animals. Mutation in these genes may be not lethal to seeds, but affect seed dispersal. In maize, mutation affecting starch synthesis in seeds does not affect the embryo development, but the seed germination rate is lower in mutants than that in the wild type plants (R, C, Styer and D, J, Cantliffe, Plant Physiol. 76, 196-200, 1984). In alfalfa, some lipids on pollen wall are used to attract insects and help pollen to stick to insects for pollination. Disrupting biosynthesis of these lipids may not be lethal to pollen, but affect pollen dispersal. The inventors here have found that disrupting starch biosysnthesis in pollen does not affect the normal pollen development, but renders transgenic pollen less able to compete against non-transgenic pollen to achieve fertilization. The difference between lethal gene and non-lethal gene approaches is evident. In the lethal gene approach, transgenic pollen is non-viable within anthers and none of the offspring obtained from the cross using the pollen-lethal transgenic plants as the male parent contain transgenes. However, in non-lethal gene approaches, transgenic pollen is still viable within anther and a very small number of the progenies obtained using the non-pollen-lethal transgenic plants as the male parent still contain transgenes (“escapes”). In most transgenic events, the transgene transmission rates through pollen in the non-pollen-lethal gene approach ranged from 1% to 0.001%. To eliminate these transgenic “escape” plants, a screenable color marker gene is needed, so that transgenic “escape” seeds can been sorted out using color sorting machines. Through multiple seed-sortings, less than 10-8% transgene escape rate via pollen can be achieved. To achieve seed purity for commercial hybrid seed production, this invention uses multiple components containing both non-pollen-lethal genes and screenable seed color marker genes.

The pollen-suicide and pollen-lethal gene approaches have been proposed to control transgenic polynucleotide of interest flow through pollen (US20020144305 A1,) and to propagate recessive male sterile mutants (WO93/25695, Williams, Trends Biotechnol. 13, 344-349, 1995; E. Perez-Prat, M. M. van Lookeren Campagne, Trends Plant Sci., 7, 199-203, 2002.), using cytotoxic genes (Diphtheria toxin A chain and Bacillus amyloliquefaciens Barnase). The expression of cytotoxic genes in pollen from transgenic plants not only causes pollen death but also disadvantageously results in transgenic plants with small flowers, complete male sterility and reduced female transmission that negatively affects the agronomic performance of crop plants. (D. Twell, Protoplasma. 187, 144-154, 1995. X. Zhan, H. Wu, A. Y. Cheung, Sex. Plant Reprod. 9, 35-43, 1996). This can result from the non-pollen-specific expression of the cytotoxic gene in the transgenic plants such as where a pollen specific promoter is used and expression is “leaky” where some expression occurs outside the pollen cells. Without wishing to be bound by this theory, the inventors believe that using pollen-specific or leaky pollen specific expression of the non-toxic pollination-disruption polynucleotides, rather than non-pollen-specific expression of cytotoxic genes, will result in a plant with a normal phenotype and normal or increased agronomic performance, for example, as compared to a plant that does not contain the pollination-disruption construct.

As described, the pollination-disruption construct is not toxic to plant cells, so that pollen transgenic for the pollination-disruption construct persist, for example, within the anthers but are not formed or do not function in a manner to effect pollination. In one aspect, pollen containing the pollination-disruption construct may be unable to germinate and/or to produce pollen tubes. For example, if the pollination-disruption polynucleotide encodes a starch degradation enzyme, its specific expression in pollen may disrupt starch accumulation in pollen, thereby decreasing the energy source for pollen germination and pollen tube growth and ultimately inhibiting fertilization.

Accordingly, polynucleotides that encode proteins that degrade starch and/or prevent pollen from accumulating starch and/or synthesizing starch may also be suitable for use as a pollination-disruption polynucleotide. Polynucleotides that encode starch degradation proteins are non-cytotoxic and expressed in photosynthesis tissues (A. M. Smith, S. C. Zeeman and S. M. Smith, Annu. Rev. Plant. Biol. 2005, 56:73-98), developing kernels, germinating seeds and germinating pollen grains, and are also present in human saliva. Other transgenic polynucleotides of interest include but are not limited to those that alter carbohydrates, for example, by altering a gene for an enzyme that affects the branching pattern of starch or a gene altering thioredoxin such as NTR and/or TRX (see U.S. Pat. No. 6,531,648) and/or a gamma zein knock out or mutant such as cs27 or TUSC27 or en27 (See U.S. Pat. No. 6,858,778 and US2005/0160488, US2005/0204418). See also Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtilis levansucrase gene), Pen et al., Bio/Technology 10: 292 (1992) (production of transgenic plants that express alpha-amylase), Elliot et al., Plant Molec. Biol. 21: 515 (1993) (nucleotide sequences of tomato invertase genes), Søgaard et al., J. Biol. Chem. 268: 22480 (1993) (site-directed mutagenesis of barley alpha-amylase gene), and Fisher et al., Plant Physiol. 102: 1045 (1993) (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch and oil contents and/or composition through the interrelationship of the starch and oil pathways. Further examples of proteins encoded by pollination-disruption polynucleotides include without limitation alpha-, beta-amylase gene, debranching enzymes, such as Sugary1 and pullulanase, glucanase, and SacB, and lipases. The increase or decrease in the amount of starch in a pollen containing a pollination-disruption polynucleotide may be determined using any number of methods, including dyes that stain starch, such as KI-I₂.

One skilled in the art would be familiar with a number of nucleotide sequences that encode proteins that inhibit pollen function, such as fertilization and germination, without toxicity to pollen and/or other plant cells. Other potential pollination-disruption polynucleotides or proteins may be identified using routine techniques such as gene shuffling and artificial mutagenesis. (J. E. Ness et al., Nature Biotechnol. 20, 1251, 2002.)

Thus, according to one aspect, pollination-disruption polynucleotides include any polynucleotide that is not lethal to the pollen or plant cell but can render the pollen malfunctional, for example, unable to germinate, to produce pollen tube, or to release functional sperm cells. Pollination-disruption polynucleotides or proteins may be from any source, including those isolated from organisms, such as maize, or those synthesized. In one aspect the pollination-disruption construct is operably linked to a leader sequence that directs the peptide encoded by the pollination-disruption polynucleotide into the pollen or to specific compartments within the pollen or pollen organelle, for example, the amyloplastid, mitochondria, protein bodies, oil bodies or other compartments in pollen for storing energy sources or enzymes that are critical for pollen re-hydration, germination, pollen tube growth, sperm cell release, or fertilization.

Any leader sequence may be used so long as it delivers the protein encoded by the pollination-disruption polynucleotide into a location within the pollen so that the polynucleotide or protein renders the pollen malfunctional. Thus, the term leader sequence also includes any sequences such as a signal peptide sequences or transit peptides that direct the protein to the appropriate location in the pollen. When using a starch disruption polynucleotide as the pollination disruption polynucleotide, since most crops synthesize and store starch in the amyloplast of pollen, it is thus preferred to include in the construct a leader sequence that delivers the starch disrupter to the amyloplast. For example, an alpha amylase protein may be delivered into an amyloplast using the leader sequence from the (bt) brittle 1 gene. Leader sequences from genes other than brittle 1 may be used and include but are not limited to genes imported into chloroplast and amyloplast such as prSS and prCAB (see K. Keegstra and L J. Olsen, Annu. Rev. Plant Physio. Plant Mol. Biol. 1989, 40:471-501). Sequences include those synthesized or isolated from any precursors that are targeted into the plastids, for example, chloroplasts and amyloplasts. In one aspect, the length of the transit peptide varies from 29 amino acids to nearly 100 amino acids. Generally, (leader sequences) transit peptides are rich in the hydroxylated amino acids, for example, serine and threonine, and rich in small hydrophobic amino acids such as alanine and valine. Transit peptide or leader sequences direct the transgenic polynucleotide product of interest to the chloroplasts or other plastids. Such transit peptides are known in the art. See, for example, Von Heijne et al. (1991) Plant Mol. Biol. Rep. 9:104 126; Clark et al. (1989) J. Biol. Chem. 264:17544 17550; Della-Cioppa et al. (1987) Plant Physiol. 84:965 968; Romer et al. (1993) Biochem. Biophys. Res. Commun. 196:1414 1421; and Shah et al. (1986) Science 233:478 481. In most plant crops, starch synthesis and storage occurs in the amyloplast region of the pollen, and thus it is preferred that the starch disruption encoding gene expression is preferentially directed to the amyloplast. For those plants, a leader sequence which directs expression to the amyloplast is important in achieving disruption of the pollen expression without lethal expression.

In one aspect, the pollination-disruption construct including the pollen-specific promoter operably linked to the pollination-disruption polynucleotide and/or leader sequence may be linked to one or more transgenic polynucleotides of interest. Transgenic polynucleotides of interest include but are not limited to those which impact plant insecticide resistance, disease resistance, herbicide resistance, nutrition and cellulose content, male sterility, abiotic stress resistance, for example, nitrogen fixation, yield enhancement genes, drought tolerance genes, cold tolerance genes, antibiotic resistance, genes complementing recessive agronomic traits such as recessive male sterility, and/or other marker genes.

Transgenic polynucleotides that confer resistance to insects or disease include but are not limited to the following: Bacillus thuringiensis protein, a derivative thereof or a synthetic polypeptide modeled thereon. See, for example, Geiser et al., Gene 48: 109 (1986), who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and patent applications and hereby are incorporated by reference: U.S. Pat. Nos. 5,188,960; 5,689,052; 5,880,275; WO 91/14778; WO 99/31248; WO 01/12731; WO 99/24581; WO 97/40162 and U.S. application Ser. Nos. 10/032,717; 10/414,637; and 10/606,320; an insect-specific hormone or pheromone such as an ecdysteroid and juvenile hormone, a variant thereof, a mimetic based thereon, or an antagonist or agonist thereof. See, for example, the disclosure by Hammock et al., Nature 344: 458 (1990), of baculovirus expression of cloned juvenile hormone esterase, an inactivator of juvenile hormone; an insect-specific peptide which, upon expression, disrupts the physiology of the affected pest. For example, see the disclosures of Regan, J. Biol. Chem. 269: 9 (1994) (expression cloning yields DNA coding for insect diuretic hormone receptor); Pratt et al., Biochem. Biophys. Res. Comm. 163: 1243 (1989) (an allostatin is identified in Diploptera puntata); Chattopadhyay et al. (2004) Critical Reviews in Microbiology 30 (1): 33-54 2004; Zjawiony (2004) J Nat Prod 67 (2): 300-310; Carlini & Grossi-de-Sa (2002) Toxicon, 40 (11): 1515-1539; Ussuf et al. (2001) Curr Sci. 80 (7): 847-853; and Vasconcelos & Oliveira (2004) Toxicon 44 (4): 385-403. See also U.S. Pat. No. 5,266,317 to Tomalski et al., who disclose genes encoding insect-specific toxins; an enzyme responsible for a hyperaccumulation of a monoterpene, a sesquiterpene, a steroid, hydroxamic acid, a phenylpropanoid derivative or another non-protein molecule with insecticidal activity; an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23: 691 (1993), who teach the nucleotide sequence of a cDNA encoding tobacco hookworm chitinase, and Kawalleck et al., Plant Molec. Biol. 21: 673 (1993), who provide the nucleotide sequence of the parsley ubi4-2 polyubiquitin gene, U.S. application Ser. Nos. 10/389,432, 10/692,367, and U.S. Pat. No. 6,563,020; a molecule that stimulates signal transduction. For example, see the disclosure by Botella et al., Plant Molec. Biol. 24: 757 (1994), of nucleotide sequences for mung bean calmodulin cDNA clones, and Griess et al., Plant Physiol. 104: 1467 (1994), who provide the nucleotide sequence of a maize calmodulin cDNA clone; a hydrophobic moment peptide. See PCT application WO 95/16776 and U.S. Pat. No. 5,580,852 (disclosure of peptide derivatives of Tachyplesin which inhibit fungal plant pathogens) and PCT application WO 95/18855 and U.S. Pat. No. 5,607,914) (teaches synthetic antimicrobial peptides that confer disease resistance); a membrane permease, a channel former or a channel blocker. For example, see the disclosure by Jaynes et al., Plant Sci. 89: 43 (1993), of heterologous expression of a cecropin-beta lytic peptide analog to render transgenic tobacco plants resistant to Pseudomonas solanacearum; a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28: 451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus; an insect-specific antibody or an immunotoxin derived therefrom. Thus, an antibody targeted to a critical metabolic function in the insect gut would inactivate an affected enzyme, killing the insect. Cf. Taylor et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments); a virus-specific antibody. See, for example, Tavladoraki et al., Nature 366: 469 (1993), who show that transgenic plants expressing recombinant antibody genes are protected from virus attack; a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See Lamb et al., Bio/Technology 10: 1436 (1992). The cloning and characterization of a gene which encodes a bean endopolygalacturonase-inhibiting protein is described by Toubart et al., Plant J. 2: 367 (1992); a developmental-arrestive protein produced in nature by a plant. For example, Logemann et al., Bio/Technology 10: 305 (1992), have shown that transgenic plants expressing the barley ribosome-inactivating gene have an increased resistance to fungal disease; genes involved in the Systemic Acquired Resistance (SAR) Response and/or the pathogenesis related genes. Briggs, S., Current Biology, 5(2):128-131 (1995), Pieterse & Van Loon (2004) Curr. Opin. Plant Bio. 7(4):456-64 and Somssich (2003) Cell 113(7):815-6; Antifungal genes (Cornelissen and Melchers, Pl. Physiol. 101:709-712, (1993) and Parijs et al., Planta 183:258-264, (1991) and Bushnell et al., Can. J. of Plant Path. 20(2):137-149 (1998). Also see U.S. Pat. No. 6,875,907; Detoxification genes, such as for fumonisin, beauvericin, moniliformin and zearalenone and their structurally related derivatives. For example, see U.S. Pat. No. 5,792,931; Cystatin and cysteine proteinase inhibitors. See U.S. application Ser. No. 10/947,979; Defensin genes. See WO03000863 and U.S. application Ser. No. 10/178,213; Genes conferring resistance to nematodes. See WO 03/033651 and Urwin et. al., Planta 204:472-479 (1998), Williamson (1999) Curr Opin Plant Bio. 2(4):327-31; Genes such as rcg1 conferring resistance to Anthracnose stalk rot, which is caused by the fungus Colletotrichum graminiola. See M. Jung et al., Generation-means analysis and quantitative trait locus mapping of Anthracnose Stalk Rot genes in Maize, Theor. Appl. Genet. (1994) 89:413-418 which is incorporated by reference for this purpose, as well as U.S. Patent Application 60/675,664, which is also incorporated by reference. Transgenic polynucleotides that confer resistance to insects also include those that provide resistance to striga, for example, Bt.

Transgenic polynucleotides of interest that confer resistance to a herbicide, include without limitation, a herbicide that inhibits the growing point or meristem, such as an imidazolinone or a sulfonylurea. Exemplary genes in this category code for mutant ALS and AHAS enzyme as described, for example, by Lee et al., EMBO J. 7: 1241 (1988), and Miki et al., Theor. Appl. Genet. 80: 449 (1990), respectively. See also, U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference for this purpose; glyphosate (resistance imparted by mutant 5-enolpyruv1-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cycloshexones (ACCase inhibitor-encoding genes). See, for example, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications EP1173580; WO 01/66704; EP1173581 and EP1173582, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. See, for example, U.S. Application Serial Nos. US01/46227; 10/427,692 and 10/427,692. A DNA molecule encoding a mutant aroA gene can be obtained under ATCC accession No. 39256, and the nucleotide sequence of the mutant gene is disclosed in U.S. Pat. No. 4,769,061 to Comai. European Patent Application No. 0 333 033 to Kumada et al. and U.S. Pat. No. 4,975,374 to Goodman et al. disclose nucleotide sequences of glutamine synthetase genes which confer resistance to herbicides such as L-phosphinothricin. The nucleotide sequence of a phosphinothricin-acetyl-transferase gene is provided in European Patent No. 0 242 246 and 0 242 236 to Leemans et al. De Greef et al., Bio/Technology 7: 61 (1989), describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1; and 5,879,903, which are incorporated herein by reference for this purpose. Exemplary genes conferring resistance to phenoxy proprionic acids and cycloshexones, such as sethoxydim and haloxyfop, are the Acc1-S1, Acc1-S2 and Acc1-S3 genes described by Marshall et al., Theor. Appl. Genet. 83: 435 (1992). A herbicide that inhibits photosynthesis, such as a triazine (psbA and gs+ genes) and a benzonitrile (nitrilase gene) is also included. Przibilla et al., Plant Cell 3: 169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285: 173 (1992). Acetohydroxy acid synthase, which has been found to make plants that express this enzyme resistant to multiple types of herbicides, has been introduced into a variety of plants (see, e.g., Hattori et al. (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota et al. (1994) Plant Physiol. 106(1):17-23), genes for glutathione reductase and superoxide dismutase (Aono et al. (1995) Plant Cell Physiol 36:1687, and genes for various phosphotransferases (Datta et al. (1992) Plant Mol Biol 20:619). Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1; and 5,767,373; and international publication WO 01/12825.

Transgenic polynucleotides of interest also include those genes that confer or contribute to nutrition, cellulose content, or alter grain characteristic, such as an altered fatty acid, for example, by down-regulation of stearoyl-ACP desaturase to increase stearic acid content of the plant. See Knultzon et al., Proc. Natl. Acad. Sci. USA 89: 2624 (1992) and WO99/64579 (Genes for Desaturases to Alter Lipid Profiles in Corn), elevating oleic acid via FAD-2 gene modification and/or decreasing linolenic acid via FAD-3 gene modification (see U.S. Pat. Nos. 6,063,947; 6,323,392; 6,372,965 and WO 93/11245); altering conjugated linolenic or linoleic acid content, such as in WO 01/12800; altering LEC1, AGP, Dek1, Superal1,mi1ps, various lpa genes such as lpa1, Ipa3, hpt or hggt. For example, see WO 02/42424, WO 98/22604, WO 03/011015, U.S. Pat. No. 6,423,886, U.S. Pat. No. 6,197,561, U.S. Pat. No. 6,825,397, US2003/0079247, US2003/0204870, WO02/057439, WO03/011015 and Rivera-Madrid, R. et. al. Proc. Natl. Acad. Sci. 92:5620-5624 (1995). Transgenic polynucleotides of interest also include those that alter phosphorus content, for example, by the introduction of a phytase-encoding gene that would enhance breakdown of phytate, adding more free phosphate to the transformed plant. For example, see Van Hartingsveldt et al., Gene 127: 87 (1993), for a disclosure of the nucleotide sequence of an Aspergillus niger phytase gene; or those that up-regulate a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy et al., Maydica 35: 383 (1990) and/or by altering inositol kinase activity as in WO 02/059324, US2003/0009011, WO 03/027243, US2003/0079247, WO 99/05298, U.S. Pat. No. 6,197,561, U.S. Pat. No. 6,291,224, U.S. Pat. No. 6,391,348, WO2002/059324, US2003/0079247, Wo98/45448, WO99/55882, WO01/04147. Other transgenic polynucleotides of interest include but are not limited to those that alter carbohydrates effected, for example, by altering a gene for an enzyme that affects the branching pattern of starch or a gene altering thioredoxin such as NTR and/or TRX (see U.S. Pat. No. 6,531,648 which is incorporated by reference for this purpose) and/or a gamma zein knock out or mutant such as cs27 or TUSC27 or en27 (See U.S. Pat. No. 6,858,778 and US2005/0160488, US2005/0204418; which are incorporated by reference for this purpose). See Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtilis levansucrase gene), Pen et al., Bio/Technology 10: 292 (1992) (production of transgenic plants that express Bacillus licheniformis alpha-amylase), Elliot et al., Plant Molec. Biol. 21: 515 (1993) (nucleotide sequences of tomato invertase genes), Søgaard et al., J. Biol. Chem. 268: 22480 (1993) (site-directed mutagenesis of barley alpha-amylase gene), and Fisher et al., Plant Physiol. 102: 1045 (1993) (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

Transgenic polynucleotides of interest also include those genes that confer or contribute to an altered grain characteristic include without limitation the those that alter antioxidant content or composition, such as alteration of tocopherol or tocotrienols. For example, see U.S. Pat. No. 6,787,683, US2004/0034886 and WO 00/68393 involving the manipulation of antioxidant levels through alteration of a phyt1 prenyl transferase (ppt), WO 03/082899 through alteration of a homogentisate geranyl geranyl transferase (hggt).

Also included are those that alter essential seed amino acids. For example, see U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US2003/0163838, US2003/0150014, US2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US2004/0025203 (UDPGdH), U.S. Pat. No. 6,194,638 (RGP).

Transgenic polynucleotides of interest also include but are not limited to genes that control male-sterility. There are several methods of conferring genetic male sterility available, such as multiple mutant genes at separate locations within the genome that confer male sterility, as disclosed in U.S. Pat. Nos. 4,654,465 and 4,727,219 to Brar et al. and chromosomal translocations as described by Patterson in U.S. Pat. Nos. 3,861,709 and 3,710,511. In addition to these methods, Albertsen et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on”, the promoter, which in turn allows the gene that confers male fertility to be transcribed. Male sterility is also affected by the introduction of various transgenes for example, introduction of a deacetylase gene under the control of a tapetum-specific promoter and with the application of the chemical N-Ac-PPT (WO 01/29237), or of various stamen-specific promoters (WO 92/13956, WO 92/13957), or of the barnase and the barstar gene (Paul et al. Plant Mol. Biol. 19:611-622, 1992). For additional examples of nuclear male and female sterility systems and genes, see also, U.S. Pat. Nos. 5,859,341; 6,297,426; 5,478,369; 5,824,524; 5,850,014; and 6,265,640; all of which are hereby incorporated by reference.

Other transgenic polynucleotides of interest include but are not limited to genes that create a site for site specific DNA integration. This includes, for example, the introduction of FRT sites that may be used in the FLP/FRT system and/or Lox sites that may be used in the Cre/Loxp system. For example, see Lyznik, et al., Site-Specific Recombination for Genetic Engineering in Plants, Plant Cell Rep (2003) 21:925-932 and WO 99/25821, which are hereby incorporated by reference. Other systems that may be used include the Gin recombinase of phage Mu (Maeser et al., 1991; Vicki Chandler, The Maize Handbook ch. 118 (Springer-Verlag 1994), the Pin recombinase of E. coli (Enomoto et al., 1983), and the R/RS system of the pSR1 plasmid (Araki et al., 1992).

Transgenic polynucleotides of interest also include but are not limited to genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US2004/0148654 and WO01/36596 where abscisic acid is altered in plants resulting in improved plant phenotype such as increased yield and/or increased tolerance to abiotic stress; WO2000/006341, WO04/090143, U.S. application Ser. Nos. 10/817,483 and 09/545,334 where cytokinin expression is modified resulting in plants with increased stress tolerance, such as drought tolerance, and/or increased yield. Also see WO0202776, WO2003052063, JP2002281975, U.S. Pat. No. 6,084,153, WO0164898, U.S. Pat. No. 6,177,275, and U.S. Pat. No. 6,107,547 (enhancement of nitrogen utilization and altered nitrogen responsiveness). For ethylene alteration, see US20040128719, US20030166197 and WO200032761. For plant transcription factors or transcriptional regulators of abiotic stress, see e.g. US20040098764 or US20040078852.

Other genes and transcription factors that affect plant growth and agronomic traits such as yield, flowering, plant growth and/or plant structure, can be introduced or introgressed into plants, see e.g. WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. No. 6,794,560, U.S. Pat. No. 6,307,126 (GAI), WO99/09174 (D8 and Rht), and WO2004076638 and WO2004031349 (transcription factors).

Transgenic polynucleotides of interests of interest also include but are not limited to marker genes. A marker provides a means for screening a population of organisms or cells of an organism (e.g., plants or plant cells) to identify those having the marker and, therefore, the transgenic polynucleotide of interest. Also for with respect to non-food crops, a fluorescent protein may be preferred marker with which to facilitate selection. A selectable marker may confers a selective advantage to the cell, or to an organism (e.g., a plant) containing the cell, for example, the ability to grow in the presence of a negative selective agent such as an antibiotic or, for a plant, an herbicide. A selective advantage also can be due, for example, to an enhanced or novel capacity to utilize an added compound as a nutrient, growth factor or energy source. One such example is provitamin A or Beta-carotene. (Ye, X et al., Science 287: 303-305, 2000; M. Schledz et al., Plant J. 10:781-792, 1996; M. Bonk et al., Eur. J. Biochem. 247: 942, 1997; N. Misawa, et. al., Plant J. 4: 833, 1993. Gene sequence information: a plant phytoene synthase (psy) originating from daffodil, GeneBank accession number X78814; a baterial phytoene desaturase (crtI) originating from Erwinia uredovora, GeneBank accession number D90087; lycopene β-cyclase from Narcissus pseudonarcissus, GeneBank accession number X98796) A selective advantage can be conferred by a single polynucleotide, or its expression product, or by a combination of polynucleotides whose expression in a plant cell gives the cell a positive selective advantage, a negative selective advantage, or both. It should be recognized that expression of the transgenic polynucleotide of interest (e.g., encoding a hpRNA) also provides a means to select cells containing the encoding nucleotide sequence. However, the use of an additional selectable marker, which, for example, allows a plant cell to survive under otherwise toxic conditions, provides a means to enrich for transformed plant cells containing the desired transgenic polynucleotide of interest.

Examples of selectable markers include those that confer resistance to antimetabolites such as herbicides or antibiotics, for example, dihydrofolate reductase, which confers resistance to methotrexate (Reiss, Plant Physiol. (Life Sci. Adv.) 13:143-149, 1994; see also Herrera Estrella et al., Nature 303:209-213, 1983; Meijer et al., Plant Mol. Biol. 16:807-820, 1991); neomycin phosphotransferase, which confers resistance to the aminoglycosides neomycin, kanamycin and paromycin (Herrera-Estrella, EMBO J. 2:987-995, 1983) and hygro, which confers resistance to hygromycin (Marsh, Gene 32:481-485, 1984; see also Waldron et al., Plant Mol. Biol. 5:103-108, 1985; Zhijian et al., Plant Science 108:219-227, 1995); trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include, for example, a mutant EPSPV-synthase, which confers glyphosate resistance (Hinchee et al., BioTechnology 91:915-922, 1998), a mutant acetolactate synthase, which confers imidazolinone or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., EMBO J. 2:987-992, 1983); streptomycin (Jones et al., Mol. Gen. Genet. 210:86-91, 1987); spectinomycin (Bretagne-Sagnard et al., Transgenic Res. 5:131-137, 1996); bleomycin (Hille et al., Plant Mol. Biol. 7:171-176, 1990); sulfonamide (Guerineau et al., Plant Mol. Biol. 15:127-136, 1990); bromoxynil (Stalker et al., Science 242:419-423, 1988); glyphosate (Shaw et al., Science 233:478-481, 1986); phosphinothricin (DeBlock et al., EMBO J. 6:2513-2518, 1987), and the like. One option for use of a selective gene is a glufosinate-resistance encoding DNA and in one embodiment can be the phosphinothricin acetyl transferase (“PAT”), maize optimized PAT gene or bar gene under the control of the CaMV 35S or ubiquitin promoters. The genes confer resistance to bialaphos. See, Gordon-Kamm et al., Plant Cell 2:603; 1990; Uchimiya et al., BioTechnology 11:835, 1993; White et al., Nucl. Acids Res. 18:1062, 1990; Spencer et al., Theor. Appl. Genet. 79:625-631, 1990; and Anzai et al., Mol. Gen. Gen. 219:492, 1989). A version of the PAT gene is the maize optimized PAT gene, described at U.S. Pat. No. 6,096,947.

In addition, markers that facilitate identification of a plant cell containing the polynucleotide encoding the marker may be employed. Scorable or screenable markers are useful, where presence of the sequence produces a measurable product. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. The EMBO Journal vol. 6 No. 13 pp. 3901-3907); alkaline phosphatase. In a preferred embodiment, the marker used is beta-carotene or provitamen A (Ye et al, supra). The gene has been used to enhance the nutrition of rice, but in this instance it is employed instead as a screenable marker, and the presence of the gene linked to a gene of interest is detected by the golden color provided. Unlike the situation where the gene is used for its nutritional contribution to the plant, a smaller amount of the protein is needed. Other screenable markers include the anthocyanin/flavonoid genes in general (See discussion at Taylor and Briggs, The Plant Cell (1990)2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., Plant Cell (1996) 8: 1171-1179; Scheffler et al. Mol. Gen. Genet. (1994) 242:40-48) and maize C2 (Wienand et al., Mol. Gen. Genet. (1986) 203:202-207); the B gene (Chandler et al., Plant Cell (1989) 1:1175-1183), the p1 gene (Grotewold et al, Proc. Natl. Acad. Sci USA (1991) 88:4587-4591; Grotewold et al., Cell (1994) 76:543-553; Sidorenko et al., Plant Mol. Biol. (1999)39:11-19); the bronze locus genes (Ralston et al., Genetics (1988) 119:185-197; Nash et al., Plant Cell (1990) 2(11): 1039-1049), among others. Yet further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PhiYFP™ from Evrogen; see Bolte et al. (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al. (2002) Biotechniques 2(2):286-293). Additional examples include a p-lactamase gene (Sutcliffe, Proc. Nat'l. Acad. Sci. U.S.A. (1978) 75:3737), which encodes an enzyme for which various chromogenic substrates are known (e.g., PADAC, a chromogenic cephalosporin); a xylE gene (Zukowsky et al., Proc. Nat'l. Acad. Sci. U.S.A. (1983) 80:1101), which encodes a catechol dioxygenase that can convert chromogenic catechols; an α-amylase gene (Ikuta et al., Biotech. (1990) 8:241); and a tyrosinase gene (Katz et al., J. Gen. Microbiol. (1983) 129:2703), which encodes an enzyme capable of oxidizing tyrosine to DOPA and dopaquinone, which in turn condenses to form the easily detectable compound melanin. Clearly, many such markers are available to one skilled in the art.

The promoter for driving expression of the transgenic polynucleotide of interest may be selected based on a number of criteria, including but not limited to what the desired use is for the transgenic polynucleotide of interest, what location in the plant is expression of the transgenic polynucleotide of interest desired, and at what level is expression of transgenic polynucleotide of interest desired or whether it needs to be controlled in another spatial or temporal manner. For example, if the transgenic polynucleotide of interest is to be used to separate transgenic seed from non-transgenic seed, a non lethal marker such as a visually scorable color marker that expresses at detectable, preferably high levels, in the seed may be desirable. Any promoter that can express the color markers in seeds may be used. In one aspect, a promoter that directs expression to particular tissue may be desirable. When referring to a promoter that directs expression to a particular tissue is meant to include promoters referred to as tissue specific or tissue preferred. Included within the scope of the invention are promoters that express highly in the plant tissue, express more in the plant tissue than in other plant tissue, or express exclusively in the plant tissue. For example, “seed-specific” promoters may be employed to drive expression of a color marker. Specific-seed promoters include those promoters active during seed development, promoters active during seed germination, and/or that are expressed only in the seed. Seed-specific promoters, such as annexin, P34, β-phaseolin, α subunit of β-conglycinin, oleosin, zein, napin promoters have been identified in many plant species such as maize, wheat, rice and barley. See U.S. Pat. Nos. 7,157,629, 7,129,089, and 7,109,392. Such seed-preferred promoters further include, but are not limited to, Cim1 (cytokinin-induced message); cZ19B1 (maize 19 kDa zein); and milps (myo-inositol-1-phosphate synthase); (see WO 00/11177, herein incorporated by reference). The 27 kDa gamma-zein promoter is a preferred endosperm-specific promoter. The maize globulin-1 and oleosin promoters are preferred embryo-specific promoters. For dicots, seed-specific promoters include, but are not limited to, bean β-phaseolin, napin, β-conglycinin, soybean lectin, cruciferin, and the like. For monocots, seed-specific promoters include, but are not limited to, promoters of the 15 kDa beta-zein, 22 kDa alpha-zein, 27 kDa gamma-zein, waxy, shrunken 1, shrunken 2, globulin 1, an LtpI, an Ltp2, and oleosin genes. See also WO 00/12733, where seed-preferred promoters from end1 and end2 genes are disclosed; herein incorporated by reference. Any suitable promoter can be used that directs expression of the transgene of interest, including, for example, a constitutively active promoter such as an ubiquitin promoter, which generally effects transcription in most or all plant cells.

For example, if the transgenic polynucleotide of interest is a male or female fertility or sterility gene a promoter that expresses at detectable levels in the plant female and male cells may be desirable, and is discussed supra

The promoter may be a “female-preferential” promoter that has transcriptional activity only in or primarily in one or more of the cells or tissues of a female reproductive structure of a plant, for example, a carpel, or gynoecium (pistil), stigma, style, ovary, and cells or tissues which comprise the stigma, style and ovary. Female-preferential promoters useful in the present invention in plants include but are not limited to, dicot promoters such as a modified S13 promoter (Dzelkalns et al., Plant Cell 5:855 (1993)), the Stig1 promoter of tobacco (Goldman et al., EMBO J. 13:2976-2984 (1994)), the AGL5 promoter (Savidge et al., Plant Cell 7:721-733 (1995)), and the promoter from tobacco TTS1 (Cheung et al., Cell 82:383-393 (1995)). The above promoters have all been tested and shown to be functional in transgenic plants. Monocot derived promoters include the promoter of the maize carpel-specific ZAG2 gene (Thiessen et al., Gene 156:155-166 (1995)). Additionally, genomic DNA containing promoter sequences can be isolated which correspond to a cDNA known in the art to have female preferential expression. These include, but are not limited to, promoters for the Arabidopsis Fbp7 and Fbp11 genes (Angenent et al., Plant Cell 7:1569-1582 (1995)) and the orchid female-specific cDNAs O40, O108, O39, O126 and O141 (Nadeau et al., Plant Cell 8:213-239 (1996)).

Promoters useful for expressing a nucleic acid molecule of interest can be any of a range of naturally-occurring promoters known to be operative in plants or animals, as desired. Promoters that direct expression in cells of male or female reproductive organs of a plant are useful for generating a transgenic plant or breeding pair of plants of the invention. For example, the promoter for male sterility genes may be their own promoters or any promoter the can express the fertility gene to restore male fertility to male sterile plants. Thus, the promoter may be homologous or heterologous with respect to the transgenic polynucleotide of interest to be expressed.

The promoters useful in the present invention can include constitutive promoters, which generally are active in most or all tissues of a plant; inducible promoters, which generally are inactive or exhibit a low basal level of expression, and can be induced to a relatively high activity upon contact of cells with an appropriate inducing agent; tissue-specific (or tissue-preferred) promoters, which generally are expressed in only one or a few particular cell types (e.g., plant anther cells); and developmental- or stage-specific promoters, which are active only during a defined period during the growth or development of a plant. Often promoters can be modified, if necessary, to vary the expression level. Certain embodiments comprise promoters exogenous to the species being manipulated. For example, the Ms45 gene introduced into ms45ms45 maize germplasm may be driven by a promoter isolated from another plant species; a hairpin construct may then be designed to target the exogenous plant promoter, reducing the possibility of hairpin interaction with non-target, endogenous maize promoters.

Exemplary constitutive promoters include the 35S cauliflower mosaic virus (CaMV) promoter (Odell et al. (1985) Nature 313:810-812), the maize ubiquitin promoter (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); ALS promoter (U.S. Pat. No. 5,659,026); rice actin promoter (U.S. Pat. No. 5,641,876; WO 00/70067), maize histone promoter (Brignon et al., Plant Mol Bio 22(6):1007-1015 (1993); Rasco-Gaunt et al., Plant Cell Rep. 21(6):569-576 (2003)) and the like. Other constitutive promoters include, for example, those described in U.S. Pat. Nos. 5,608,144 and 6,177,611, and PCT publication WO 03/102198.

An inducible regulatory element is one that is capable of directly or indirectly activating transcription of one or more DNA sequences or genes in response to an inducer. The inducer can be a chemical agent such as a protein, metabolite, growth regulator, herbicide or phenolic compound; or a physiological stress, such as that imposed directly by heat, cold, salt, or toxic elements, or indirectly through the action of a pathogen or disease agent such as a virus; or other biological or physical agent or environmental condition. A plant cell containing an inducible regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression. Typically the protein factor that binds specifically to an inducible regulatory element to activate transcription is present in an inactive form which is then directly or indirectly converted to the active form by the inducer. Any inducible promoter can be used in the instant invention (See Ward et al., Plant Mol. Biol. 22: 361-366, 1993).

Examples of inducible regulatory elements include a metallothionein regulatory element, a copper-inducible regulatory element, or a tetracycline-inducible regulatory element, the transcription from which can be effected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994). Inducible regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991; U.S. Pat. No. 6,504,082); a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; and the light-inducible promoter derived from the pea rbcS gene or pea psaDb gene (Yamamoto et al. (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6):1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-340). Certain promoters are inducible by wounding, including the Agrobacterium pmas promoter (Guevara-Garcia et al. (1993) Plant J. 4(3):495-505) and the Agrobacterium ORF13 promoter (Hansen et al., (1997) Mol. Gen. Genet. 254(3):337-343).

Additional regulatory elements active in plant cells and useful in the methods or compositions of the invention include, for example, the spinach nitrite reductase gene regulatory element (Back et al., Plant Mol. Biol. 17:9, 1991); a gamma zein promoter, an oleosin ole16 promoter, a globulin I promoter, an actin I promoter, an actin cl promoter, a sucrose synthetase promoter, an INOPS promoter, an EXM5 promoter, a globulin2 promoter, a b-32, ADPG-pyrophosphorylase promoter, an LtpI promoter, an Ltp2 promoter, an oleosin ole17 promoter, an oleosin ole18 promoter, an actin 2 promoter an anther specific RTS2 gene promoter, or G9 gene promoter, a tapetum specific RAB24 gene promoter, an anthranilate synthase alpha subunit promoter, an alpha zein promoter, an anthranilate synthase beta subunit promoter, a dihydrodipicolinate synthase promoter, a Thi 1 promoter, an alcohol dehydrogenase promoter, a cab binding protein promoter, an H3C4 promoter, a RUBISCO SS starch branching enzyme promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphate-1-phosphotransferase promoter, a beta-ketoacyl-ACP synthase promoter, a 33 kDa photosystem 11 promoter, an oxygen evolving protein promoter, a 69 kDa vacuolar ATPase subunit promoter, a glyceraldehyde-3-phosphate dehydrogenase promoter, an ABA- and ripening-inducible-like protein promoter, a phenylalanine ammonia lyase promoter, an adenosine triphosphatase S-adenosyl-L-homocysteine hydrolase promoter, a chalcone synthase promoter, a zein promoter, an auxin-binding protein promoter, a UDP glucose flavonoid glycosyl-transferase gene promoter, an NTI promoter, an actin promoter, and an opaque 2 promoter. The expression level from a promoter in a particular cell or tissue may be determined using any suitable method including Northern blot analysis. Promoters may be amplified, synthesized or isolated using techniques known to those skilled in the art.

In one aspect, the pollination-disruption construct may be in the form of a plasmid, a vector, a DNA fragment, bacterium, viral vector, or other delivery vehicle. In addition, expression vectors and in vitro culture methods suitable for plant cell or tissue transformation and regeneration of plants are routine and well-known (see, e.g., Gruber et al., “Vectors for Plant Transformation”; Id. at pages 89-119). The cells or plants that contain the construct or multiple constructs may be selected using any suitable marker or technology that allows for its identification or the tracking of the transgenic polynucleotide of interest. One could use any number of techniques known to one of skill in the art to track and breed for the constructs containing one or more transgenic polynucleotide of interest. For example, progeny tests, PCR, molecular markers, or ELISA could be used to trace the transgenic polynucleotides of interest. For example, quantitative PCR could be used to determine which progeny contain which construct and in what dose, and whether it was homozygous or heterozygous for the transgenic polynucleotide of interest. Any technique or combination of techniques may be used.

In one aspect, a plant cell may be transformed with a pollination-disruption construct linked to at least one transgenic polynucleotide of interest and the transformed plant cell generated into a plant. The construct may be introduced to the plant cell using any suitable method, including, but not limited to bombardment, transformation methods, Agrobacterium, silicon carbide fibers, electroporation, microinjection and the like.

One or more exogenous nucleic acid molecules can be introduced into plant cells using any of numerous well-known and routine methods for plant transformation, including biological and physical plant transformation protocols (see, e.g., Miki et al., “Procedures for Introducing Foreign DNA into Plants”; In Methods in Plant Molecular Biology and Biotechnology, Glick and Thompson, Eds. (CRC Press, Inc., Boca Raton, 1993) pages 67-88). In addition, expression vectors and in vitro culture methods for plant cell or tissue transformation and regeneration of plants are routine and well-known (see, e.g., Gruber et al., “Vectors for Plant Transformation”; Id. at pages 89-119).

Suitable methods of transforming plant cells include microinjection, Crossway et al. (1986) Biotechniques 4:320-334; electroporation, Riggs et al. (1986) Proc. Natl. Acad. Sci. USA 83:5602-5606; Agrobacterium-mediated transformation, see for example, Townsend et al. U.S. Pat. No. 5,563,055; direct gene transfer, Paszkowski et al. (1984) EMBO J. 3:2717-2722; and ballistic particle acceleration, see for example, Sanford et al. U.S. Pat. No. 4,945,050; Tomes et al. (1995) in Plant Cell, Tissue, and Organ Culture: Fundamental Methods, ed. Gamborg and Phillips (Springer-Verlag, Berlin); and McCabe et al. (1988) Biotechnology 6:923-926. Also see Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Klein et al. (1988) Plant Physiol. 91:440-444 (maize); Fromm et al. (1990) Biotechnology 8:833-839; Hooydaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. G. P. Chapman et al. (Longman, N.Y.), pp. 197-209 (pollen); Kaeppler et al. (1990) Plant Cell Reports 9:415-418; and Kaeppler et al. (1992) Theor. Appl. Genet. 84:560-566 (whisker-mediated transformation); D. Halluin et al. (1992) Plant Cell 4:1495-1505 (electroporation); Li et al. (1993) Plant Cell Reports 12:250-255 and Christou et al. (1995) Annals of Botany 75:407-413 (rice); Osjoda et al. (1996) Nature Biotechnology 14:745-750 (maize via Agrobacterium tumefaciens); all of which are herein incorporated by reference.

Plastid transformation also can be used to introduce a nucleic acid molecule, such as the pollination-disruption construct, into a plant cell (U.S. Pat. Nos. 5,451,513, 5,545,817, and 5,545,818; WO 95/16783; McBride et al., Proc. Natl. Acad. Sci., USA 91:7301-7305, 1994). Chloroplast transformation involves introducing regions of cloned plastid DNA flanking a desired nucleotide sequence, for example, a marker together with polynucleotide of interest, into a suitable target tissue, using, for example, a biolistic or protoplast transformation method (e.g., calcium chloride or PEG mediated transformation). One to 1.5 kb flanking regions (“targeting sequences”) facilitate homologous recombination with the plastid genome, and allow the replacement or modification of specific regions of the plastome. Using this method, point mutations in the chloroplast 16S rRNA and rps12 genes, which confer resistance to spectinomycin and streptomycin and can be utilized as selectable markers for transformation (Svab et al., Proc. Natl. Acad. Sci., USA 87:8526-8530, 1990; Staub and Maliga, Plant Cell 4:39-45, 1992), resulted in stable homopiasmic transformants, at a frequency of approximately one per 100 bombardments of target leaves. The presence of cloning sites between these markers allowed creation of a plastid targeting vector for introduction of foreign genes (Staub and Maliga, EMBO J. 12:601-606, 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga, Proc. Natl. Acad. Sci., USA 90:913-917, 1993). Approximately 15 to 20 cell division cycles following transformation are generally required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

Standard methods for transformation of canola are described at Moloney et al. “High Efficiency Transformation of Brassica napus using Agrobacterium Vectors” Plant Cell Reports 8:238-242 (1989). Corn transformation is described by Fromm et al, Bio/Technology 8:833 (1990) and Gordon-Kamm et al, supra. Agrobacterium is primarily used in dicots, but certain monocots such as maize can be transformed by Agrobacterium. See supra and U.S. Pat. No. 5,550,318. Rice transformation is described by Hiei et al., “Efficient Transformation of Rice (Oryza sativs L.) Mediated by Agrobacterium and Sequence Analysis of the Boundaries of the T-DNA” The Plant Journal 6(2): 271-282 (1994, Christou et al, Trends in Biotechnology 10:239 (1992) and Lee et al, Proc. Nat'l Acad. Sci. USA 88:6389 (1991). Wheat can be transformed by techniques similar to those used for transforming corn or rice. Sorghum transformation is described at Casas et al, supra and sorghum by Wan et al, Plant Physicol. 104:37 (1994). Soybean transformation is described in a number of publications, including U.S. Pat. No. 5,015,580.

Methods are known in the art for the targeted insertion of a polynucleotide at a specific location in the plant genome. In one embodiment, the insertion of the polynucleotide at a desired genomic location is achieved using a site-specific recombination system. See, for example, WO 99/25821, WO 99/25854, WO 99/25840, WO 99/25855, and WO 99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed can be grown into plants in accordance with conventional ways. See, for example, McCormick et al. (1986) Plant Cell Reports 5:81-84. These plants can then be grown and pollinated with the same transformed strain or different strains, and resulting plants having expression of the desired phenotypic characteristic can then be identified. Two or more generations can be grown to ensure that expression of the desired phenotypic characteristic is stably maintained and inherited.

Approximately half the pollen that these plants produce will be viable but malfunctional and approximately the other half will not contain the pollination-disruption construct and therefore will produce functional pollen. In one aspect, the plant cell is from a wild type plant or variety. In another aspect, the plant cell is transgenic for a trait or polynucleotide of interest prior to retransformation with the pollination-disruption construct.

Plant cells expressing the construct may be selected using any number of methods, for example, color selection with red fluorescent protein (RFP), green fluorescent protein (GFP) or yellow fluorescent protein (YFP), and plant-derived color genes, for example, anthocyanin. In another aspect, the plants cells may be generated into a plant and those plants that contain the construct identified using routine techniques, such as antibiotic selection and/or herbicide selection.

In another aspect of the method, the plant cell may be co-transformed with the pollination-disruption construct and a second recombinant construct that expresses a trait or polynucleotide of interest and/or marker. In one aspect, the trait of interest is a nutrition gene. Plant cells expressing both constructs may be selected using any number of methods and the cells generated into plants.

Transgenic seeds that produce malfunctional pollen may be produced by crossing a first parent plant that contains in its genome the pollination-disruption construct that renders the pollen malfunctional and contains or is linked to a marker with a second parent plant. The first parent plant may be fertilized with transgenic or non-transgenic pollen from any sexually compatible plant. Thus, the second parent plant may be a wild type, cultivar, inbred, hybrid, etc. Accordingly, in another aspect, the plants hemizygotic for the pollination-disruption construct are cross-pollinated with pollen from a plant transgenic for a trait or polynucleotide of interest. Approximately half of the resulting seeds will contain the pollination-disruption construct which is inherited from female gametes and approximately the other half of the seeds will not be transgenic for the pollination-disruption construct. Thus, the pollination-disruption polynucleotide and linked transgenic polynucleotide of interest can be transmitted to the next generation through the female only. The system is particularly useful, since some “leakiness” of the expression of pollination-disruption polynucleotide is tolerable, since it is not lethal.

In another aspect, the first parent plant is a male-sterile female plant. Any suitable method for conferring genetic male sterility may be utilized, including, for example, the use of multiple mutant genes at separate locations within the genome that confer male sterility, as disclosed in U.S. Pat. Nos. 4,654,465 and 4,727,219 to Brar et al. and chromosomal translocations as described by Patterson in U.S. Pat. Nos. 3,861,709 and 3,710,511. In addition to these methods, a system of nuclear male sterility developed by Albertsen et al., of Pioneer Hi-Bred, U.S. Pat. No. 5,432,068, may be employed. In the employment of such methods, a gene critical to male fertility may be employed, and any such gene may be used in the invention, as may wild-type mutants conferring sterility. By way of example, the MS45 gene (see U.S. Pat. Nos. 5,478,369; 5,850,014 and 6,265,640); the MS22 gene (see U.S. Ser. No. 11/833,363 and U.S. Ser. No. 11/833,385); and MS26 gene (also known as the MSCA1 gene, see US publication number 20060015968) are among those which can be used in the process of the invention. By way of further example, the table below lists several of known male fertility mutants or genes from Zea mays.

| GENE NAME | ALTERNATE NAME | REFERENCE |
|---|---|---|
| ms1 male sterile1 | male sterile1, ms1 | Singleton, WR and Jones, DF. |
| | | 1930. J Hered 21: 266-268 |
| ms10 male sterile10 | male sterile10, ms10 | Beadle, GW. 1932. |
| | | Genetics 17: 413-431 |
| ms11 male sterile11 | ms11, male sterile11 | Beadle, GW. 1932. |
| | | Genetics 17: 413-431 |
| ms12 male sterile12 | ms12, male sterile12 | Beadle, GW. 1932. |
| | | Genetics 17: 413-431 |
| ms13 male sterile13 | ms*-6060, male sterile13, | Beadle, GW. 1932. |
| | ms13 | Genetics 17: 413-431 |
| ms14 male sterile14 | ms14, male sterile14 | Beadle, GW. 1932. |
| | | Genetics 17: 413-431 |
| ms17 male sterile17 | ms17, male sterile17 | Emerson, RA. 1932. |
| | | Science 75: 566 |
| ms2 male sterile2 | male sterile2, ms2 | Eyster, WH. 1931. J Hered |
| | | 22: 99-102 |
| ms20 male sterile20 | ms20, male sterile20 | Eyster, WH. 1934. Genetics |
| | | of Zea mays. Bibliographia |
| | | Genetica 11: 187-392 |
| ms23 male sterile23 | : ms*-6059, ms*-6031, ms*- | West, DP and Albertsen, MC. |
| | 6027, ms*-6018, ms*-6011, | 1985. MNL 59: 87 |
| | ms35, male sterile23, ms*- | |
| | Bear7, ms23 | |
| ms24 male sterile24 | ms24, male sterile24 | West, DP and Albertsen, MC. |
| | | 1985. MNL 59: 87 |
| ms25 male sterile25 | ms*-6065, ms*-6057, | Loukides, CA; Broadwater, AH; |
| | ms25, male sterile25, ms*- | Bedinger, PA. 1995. |
| | 6022 | Am J Bot 82: 1017-1023 |
| ms27 male sterile27 | ms27, male sterile27 | Albertsen, MC. 1996. MNL |
| | | 70: 30-31 |
| ms28 male sterile28 | ms28, male sterile28 | Golubovskaya, IN. 1979. |
| | | MNL 53: 66-70 |
| ms29 male sterile29 | male sterile29, ms*-JH84A, | Trimnell, MR et al. 1998. |
| | ms29 | MNL 72: 37-38 |
| ms3 male sterile3 | Group 3, ms3, male sterile3 | Eyster, WH. 1931. J Hered |
| | | 22: 99-102 |
| ms30 male sterile30 | ms30, msx, ms*-6028, ms*- | Albertsen, MC et al. 1999. |
| | Li89, male sterile30, ms*- | MNL 73: 48 |
| | LI89 | |
| ms31 male sterile31 | ms*-CG889D, ms31, male | Trimnell, MR et al. 1998. |
| | sterile31 | MNL 72: 38 |
| ms32 male sterile32 | male sterile32, ms32 | Trimnell, MR et al. 1999. |
| | | MNL 73: 48-49 |
| ms33 male sterile33 | : ms*-6054, ms*-6024, | Patterson, EB. 1995. MNL |
| | ms33, ms*-GC89A, ms*- | 69: 126-128 |
| | 6029, male sterile6019, | |
| | Group 7, ms*-6038, ms*- | |
| | Stan1, ms*-6041, ms*- | |
| | 6019, male sterile33 | |
| ms34 male sterile34 | Group 1, ms*-6014, ms*- | Patterson, EB. 1995. MNL |
| | 6010, male sterile34, ms34, | 69: 126-128 |
| | ms*-6013, ms*-6004, male | |
| | sterile6004 | |
| ms36 male sterile36 | male sterile36 ms*-MS85A, | Trimnell, MR et al. 1999. |
| | ms36 | MNL 73: 49-50 |
| ms37 male sterile37 | ms*-SB177, ms37, male | Trimnell, MR et al. 1999. |
| | sterile 37 | MNL 73: 48 |
| ms38 male sterile38 | ms30, ms38 ms*-WL87A, | Albertsen, MC et al. 1996. |
| | male sterile38 | MNL 70: 30 |
| ms43 male sterile43 | ms43, male sterile43, ms29 | Golubovskaya, IN. 1979. Int |
| | | Rev Cytol 58: 247-290 |
| ms45 male sterile45 | Group 6, male sterile45, | Albertsen, MC; Fox, TW; |
| | ms*-6006, ms*-6040, ms*- | Trimnell, MR. 1993. Proc |
| | BS1, ms*-BS2, ms*-BS3, | Annu Corn Sorghum Ind |
| | ms45, ms45′-9301 | Res Conf 48: 224-233 |
| ms48 male sterile48 | male sterile48, ms*-6049, | Trimnell, M et al. 2002. |
| | ms48 | MNL 76: 38 |
| ms5 male sterile5 | : ms*-6061, ms*-6048, ms*- | Beadle, GW. 1932. |
| | 6062, male sterile5, ms5 | Genetics 17: 413-431 |
| ms50 male sterile50 | ms50, male sterile50, ms*- | Trimnell, M et al. 2002. |
| | 6055, ms*-6026 | MNL 76: 39 |
| ms7 male sterile7 | ms7, male sterile7 | Beadle, GW. 1932. |
| | | Genetics 17: 413-431 |
| ms8 male sterile8 | male sterile8, ms8 | Beadle, GW. 1932. |
| | | Genetics 17: 413-431 |
| ms9 male sterile9 | Group 5, male sterile9, ms9 | Beadle, GW. 1932. |
| | | Genetics 17: 413-431 |
| ms49 male sterile49 | ms*-MB92, ms49, male | Trimnell, M et al. 2002. |
| | sterile49 | MNL 76: 38-39 |

In another aspect, the male-sterile female plant is a cytoplasmic male-sterile plant. In one aspect, the first parent plant is the sorghum female plant. In another aspect, the second plant is a maintainer or restorer line. In referring to a maintainer line is meant a plant line that can maintain the male-sterile characteristic of this male sterile line, a restorer is meant a plant line that can restore the fertility of this male-sterile line. Use of a maintainer (or a restorer) line as the male parent, will produce seeds that are about half transgenic and about half non-transgenic with respect to the pollination-disruption construct. These seeds may be identified using a marker and separated as described previously.

In another aspect, methods provided herein can more efficiently propagate homozygous male sterile plants for hybrid seeds production. In one aspect, this invention utilizes naturally-occurring recessive male sterile mutants, so that their wild-type plants are all the fertility restorers. In other aspect, the male sterile mutants can be artificially created by disrupting male fertility genes using homologous recombination technology. To propagate the male sterile mutant line, a separate transgenic maintainer line may be created. The transgenic line, in a homozygous male sterile background, contains a cloned wild-type male fertility gene linked to a pollination-disruption polynucleotide and a marker gene. The cloned male fertile gene complements the male sterile mutation, allowing for the continued development of pollen. However, most pollen grains containing the transgenic polynucleotides of interest are unable to achieve fertilization due to the expression of the pollination-disruption polynucleotide, while non-transgenic pollen, carrying the sterile allele, are able to pollinate the male sterile female plants and produce a population of recessive male sterile progenies. A homogeneous male sterile population can be achieved through seeds color sorting to remove the transgenic seeds resulted from pollination by the remaining ˜0.01% transgenic pollen grains. The maintainer line is propagated by self-pollination and sorting resulting seeds for the marker gene. Since the transgenic line in this system does not transmit the transgenic polynucleotides of interest into hybrids, when using the naturally occurring male sterile mutants as the female parents, this system may be used for the production of non-transgenic hybrid seeds. The seeds may be sorted using resulting seed morphological marker genes or seed color genes allow to separate transgenic seeds and non-transgenic seeds using commercial seed sorters.

In one aspect, the pollination-disruption construct and seeds color sorting is transformed into naturally-occurring recessive male sterile mutants, such as ms45 (Albertsen et al, supra), ms26 (Loukides et al., (1995) Amer. J. Bot 82, 1017-1023) and ms22 (West and Albertsen (1985) Maize Newsletter 59:87; Neuffer et al. (1977) Mutants of maize Cold Spring Harbor laboratory Press, Cold Spring Harbor, N.Y.) from maize. The transgenic line, in a homozygous male sterile background, contains a cloned wild-type male fertility gene, such as MS45, MS26 and MS22, linked to a pollination-disruption polynucleotide, such as PG47::BT1::alpha-amylase, and a marker gene, such as LTP2::dsRED. The cloned male fertility gene complements the male sterile mutation producing pollen. However, most pollen grains containing the transgenic polynucleotides of interest are unable to achieve fertilization due to the expression of the pollination-disruption polynucleotide, while non-transgenic pollen, carrying the sterile allele, are able to achieve fertilization. When pollen grains from this transgenic line pollinate homozygous recessive male sterile plants, most of the seeds do not inherit the construct and thus contain homozygous recessive male sterile alleles, such as ms45, ms26 and ms22. About 0.01% seeds, resulted from the remaining ˜0.01% transgenic pollen still contain the construct. These transgenic seeds can be sorted out by a commercial high speed color sorter using the deRED gene as the screenable marker. Thus, a male sterile population with the seed purity standard for commercial hybrid seeds production can be produced through the combined efforts of pollination disruption genes and screenable marker genes. A maintainer line is propagated by self-pollination and seed-sorting for the marker gene. When the restorer is crossed onto the male sterile line, all pollen is viable. Thus, the progeny crop is male fertile. Any seeds that aberrantly inherit the pollination-disruption construct can be sorted out by using the marker.

In another aspect, the male sterile mutants can be artificially created by disrupting a male fertility gene using homologous recombination technology. For example, male sterile mutation may be created by targeting GAT/HRA into MS45, MS26, MS22 or any other male fertility genes. To propagate this artificially created male sterile mutant line, a maintainer line may be created as described above. The transgenic maintainer line, in a homozygous male sterile background, contains a cloned wild-type male fertility gene linked to a pollination-disruption polynucleotide, and a marker gene. The cloned male fertile gene complements the male sterile mutation but most pollen grains containing the transgenic polynucleotides of interest are unable to achieve fertilization due to the expression of the pollination-disruption polynucleotide, while non-transgenic pollen, carrying the sterile allele, are able to pollinate the male sterile female plants and produce a population with most of the plants are homozygous recessive male sterile progenies. Again, the transgenic progenies in this population should be removed through seeds sorting. The maintainer line is propagated by self-pollination and seed-sorting for the marker gene. The marker gene is also used for seed-sorting of hybrid seeds to ensure seed purity.

In one aspect, the transgenic maintainer plant uses two independent loci for an ms locus and a restorer locus to produce two different pollen types. The following is provided by way of exemplification and not limitation. One skilled in the art appreciates any number of variations are available in terms of the specific components used, such as the male sterility gene, marker, or gene of interest, and is further not limited to a particular plant. By way of example, a construct containing a glufosinate resistant gene (GAT), a sulfonylurea resistant acetolactate synthase (Hra) gene, Bacillus thuringiensis (Bt) endotoxin is inserted into the MS22, MS45, MS26 or any other male fertility locus of the plant using a targeting system, for example, homologous recombination. The resultant seeds by selfing will be segregated for male-sterile locus and pollination-disruption locus. The plants that are homozygous with respect to the transgenic polynucleotide of interest inserted into and disrupting the male-sterility gene can be identified.

After crossing the plants, the resultant seeds may be harvested. In one aspect, a mixture of the seeds of which half will contain the pollination-disruption construct and the other half will not are planted. The seeds are allowed to germinate and grow into plants. In one aspect, the plants are subjected to selection so that only the transgenic plants containing the pollination-disruption construct survive. This selection process may performed at any suitable time during the development of the plant so long as the plants containing the pollination-disruption construct survive. For example, the young plants grown from the mixed seeds, i.e. transgenic and non-transgenic seeds may be subjected to at least one herbicide or insecticide that will kill the non-transgenic plants but will allow transgenic seeds containing the construct with the corresponding herbicide or insecticide resistance gene to live.

In one aspect, the mixture of seeds may be separated, if desired. Seeds that contain transgenic polynucleotides of interest may be identified using any suitable methods or techniques. Examples include, but are not limited to, molecular marker analysis, phenotype analysis, PCR, progeny tests, molecular markers, or ELISA could be used to trace the transgenic polynucleotides of interest. For example, in one aspect, the pollination-disruption construct may contain a marker linked to the pollination-disruption polynucleotide and/or transgenic polynucleotide of interest that is a color marker, for example one encoding beta carotene, or provitamin A.

Seeds that contain the transgenic polynucleotide of interest and those seeds that do not may be identified and separated by color where seeds expressing the color marker (for example, with respect to beta carotene or provitamin A, a golden color) indicate that those seeds contain the transgenic polynucleotide of interest. In one aspect, the seeds are identified for the color marker and separated using a sorting machine. The sorting may be performed by any suitable method. For example, the transgenic seeds may be separated from non-transgenic seeds visually. This may be accomplished using a seed sorter, or using a spectrophotometer that measures a particular wavelength to separate fluorescent color markers such as green, yellow, red fluorescent protein. The transgenic and non-transgenic seeds may be distributed, sold, or planted. One may plant the genetically modified seeds that are homozygous for the transgenic polynucleotide of interest. One skilled in the art will appreciate that the plants generated from the transgenic seeds will not produce functional pollen, thereby blocking the transmission of the transgenic polynucleotide of interest into other sexually compatible plants. In one aspect, plants grown from seed produced by such a crossing may be subjected to a selection process to eliminate plants that do not contain the pollination-disruption construct linked to a transgenic polynucleotide of interest that confers antibiotic or herbicide resistance, for example, by treating the plants with the appropriate antibiotic or herbicide. Such knowledge is within the skill of one in the art.

In another aspect, the hemizygous transgenic plant, for example, turf grass, may be vegetatively propagated to yield progeny plants that are also hemizygous for the pollination-disruption construct. Although all plants generated asexually from the transgenic plants contain the transgenic polynucleotide of interest, transmission of the transgenic polynucleotide of interest via cross-pollination is eliminated because the transgenic pollen is malfunctional.

A method for developing a plant that produces malfunctional pollen in a plant breeding program using plant breeding techniques which include a plant comprising a recombinant nucleotide construct comprising a pollen-specific promoter operably linked to a pollination-disruption polynucleotide that renders the pollen malfunctional, wherein the pollen-specific promoter and the pollination-disruption polynucleotide are linked to a marker, or its parts, as a source of plant breeding material comprising: crossing the plant with a different sexually compatible plant and wherein said plant breeding techniques are selected from the group consisting of recurrent selection, backcrossing, pedigree breeding, mass selection, restriction fragment length polymorphism enhanced selection, genetic marker enhanced selection, and transformation.

Thus, provided herein are transformed plant cells produced by the methods employing a pollination-disruption construct wherein the transformed plant cells are hemizygotic for the construct. Also included are plant cells and plants produced using the methods described herein, including recombinant plant cells, hybrid, and transgenic plants comprising the constructs. More particularly, this invention provides such cells and transgenic plants which are hemizygotic for the pollination-disruption construct that produce malfunctional pollen, thereby preventing the transmission of transgenic polynucleotide of interest to other sexually compatible plants.

In one aspect, a molecular excision system may be used alter the functionality of the pollen, while ensuring that the excision enzymes are not present in the final product as indicated by markers.

Accordingly, a method for modifying the functionality of transgenic pollen is provided. The method includes crossing a first plant that is a male-sterile female plant having a first nucleic acid construct in its genome, where the construct includes a first recognition site linked to a first promoter driving expression of a first enzyme that recognizes a second recognition site linked to a second promoter driving expression of a first marker followed by the first recognition site with pollen from a second plant. In its genome, this second plant has a second nucleic acid construct where the construct comprises a second promoter that is pollen-specific linked to a second recognition site linked to a third promoter driving expression of a second enzyme linked to a second marker followed by a second recognition site linked to a pollination-disruption polynucleotide linked to all trait genes driving by their specific promoters. When both constructs are expressed in the same cell, the second enzyme cleaves the first recognition sites so that the first enzyme and first marker are excised from the genome. Likewise, the first enzyme cleaves the second recognition sites so that the pollen-specific promoter drives expression of the pollination-disruption polynucleotide linked to all the trait genes and cleaves the second recognition sites so that the second enzyme and second marker are excised from the genome. Thus, the first and second marker genes are not inherited by the progeny resulting from the cross and pollen containing the pollination-disruption polynucleotide liked to all the trait genes is malfunctional.

Any suitable pair of site-specific recombination sequences and enzymes may be used so long as the each construct does not encode a site-specific recombinase enzyme that would cleave its own recognition sites. For example, the first recognition site may be a Lox recognition site, the second recognition site may be a FRT recognition site, and the first enzyme may be a FLP enzyme, and the second enzyme may be a CRE enzyme. In another aspect, the first recognition site may be a FRT recognition site, the second recognition site may be a Lox recognition site, the first enzyme may be a CRE enzyme, and the second enzyme may be a FLP enzyme. Other corresponding recombination sites and enzymes suitable for use with the instant method include Gin/Pin and R/RS.

The site-specific recombination sequence is recognized by a recombinase enzyme, preferably selected from the group consisting of CRE, FLP, Gin and R recombinase and more preferably, the enzyme recognizing the site-specific recombination sequence is CRE recombinase.

Site-specific integrase recombinase systems have been identified in several organisms including, but not limited to, the CRE/lox system of bacteriophage P1 (Abremski et al., 1983; U.S. Pat. Nos. 4,959,317; 5,658,772), the FLP/frt system of yeast (Golic and Lindquist, 1989), the Pin recombinase of E. coli (Enomoto et al., 1983), the Gin/gix recombinase of phage Mu (Maeser et al., 1991) and the R/RS system of the pSR1 plasmid from Xygosaccharomyces rouxii (Onouchi et al., 1991; Araki et al., 1992). All of these systems have been shown to function in plants (O'Gorman et al., 1991; Maeser et al., 1991; Onouchi et al., 1991; Dale and Ow, 1991). It is believed that site-directed integration systems like CRE/lox or FLP/frt require a circular DNA intermediate. Of these systems, CRE/lox and FLP/frt have been widely utilized.

In the constructs, the enzymes and markers may be driven by any suitable promoter depending on where expression is desired and the desired level of expression. The enzymes and markers may be driven by the same or different promoters, for example, the ubiquitin promoter and/or lipid transfer protein from barley. One of ordinary skill in the art will be familiar with techniques to generate the male-sterile female and maintainer plants for generating the homozygous or hemizygous plants, including the construction of the described constructs and transformation protocols. In another aspect, the plant is a sorghum plant. In one aspect, the color marker is a fluorescent protein, including but not limited to green fluorescent protein, yellow fluorescent protein or red fluorescent protein.

Seeds that contain constructs expressing the site-specific recombinase enzymes will express the color of the color marker to yield seeds that have a different color than the seed where the site-specific recombinase enzymes were excised. Seeds that do not express the site-specific recombinase enzymes will be absent for the marker's color. Additionally, the seeds may be identified and sorted.

These plants having the pollen-specific promoter linked to second recombination site, the pollination-disruption polynucleotide and insecticide or herbicide resistance gene may be crossed with any sexually compatible plant, including one of the restorer line to produce seeds that is hemizygous for the construct. Approximately about half of the resultant seeds and pollen will be hemizygous for the construct.

Other objects, advantages and features of the present invention become apparent to one skilled in the art upon reviewing the specification and the drawings provided herein.

### EXAMPLES

### Example 1

### Cloning and Characterization of Pollen Expressed Promoters

The promoter of the pollen-specific gene PG47 of maize was provided on a cloned genomic ApaLI restriction fragment of ˜2.8 Kb, by David Lonsdale, John Innes Centre for Plant Science Research, Norwich, UK. (Allen, R. L. and Lonsdale, D. M. 1992. Sequence analysis of three members of the maize polygalacturonase gene family expressed during pollen development. Plant Molec. Biol. 20: 343-345; Allen, R. L. and Lonsdale, D. M. 1993. Molecular characterization of one of the maize polygalacturonase gene family members which are expressed during late pollen development. Plant J. 3: 261-271). An NcoI site was introduced at the translational start codon by site-directed mutagenesis (Su, T. Z. and El-Geweley, M. R. 1988. A multisite-directed mutagenesis using T7 DNA polymerase: application for reconstructing a mammalian gene. Gene 69: 81-89). The original promoter fragment comprised 2834 bp of the genomic sequence from an ApaI site up to the altered base pairs creating the NcoI site (2 bp upstream of the start codon), thus encompassing the 5′-nontranslated leader in addition to the promoter. The PG47 promoter plus 5′-nontranslated leader was joined as translational fusions to the coding sequence for 13-Glucuronidase (GUS) from Escherichia coli (Jefferson, R. 1987. Assaying chimeric genes in plants: The GUS gene fusion system. Plant Mol. Biol. Rep. 5: 387-405), followed by the 3′ nontranslated region from the Proteinase Inhibitor II gene (PinII) from Solanum tuberosum (An, G., Mitra, A., Choi, H. K., Costa, M. A., An, K., Thornburg, R. W. and Ryan, C. A. 1989. Functional analysis of the 3′ control region of the potato wound-inducible Proteinase Inhibitor II gene. Plant Cell 1: 115-122). A second chimeric construct comprised the CaMV35S (Strasbourg) supra, promoter and 3′-nontranslated region flanking a sequence encoding phosphinothricin acyltransferase (Agrevo) supra. Both chimeric plant transcription units were oriented in the same direction, with the upstream GUS construct closest to the right T-DNA border and the downstream PAT construct closest to the left T-DNA border in a T-DNA vector derived from pSB11 (T. Komari et.al. The Plant J. 10: 165-174, 1996). The constructs were designated either PHP17215 or PHP17216.

The resulting construct was introduced separately into Agrobacterium tumefaciens LBA4404(pSB1) (T. Komari et.al. The Plant J. 10: 165-174, 1996), on AB minimal medium+50 μg/ml spectinomycin following standard triparental mating with E. coli(pRK2013) (Ditta, G., Stanfield, S., Corbin, D. and Helinski, D. R. 1980. Broad host range DNA cloning system for Gram-negative bacteria: Construction of a gene bank of Rhizobium meliloti. Proc. Natl. Acad. Sci. USA 77: 7347-7351). T-DNA/Ti-plasmid cointegrates was named PHP17215.

Immature embryos from greenhouse-grown maize plants of Hi-II and Hi-II MS45EX4 are transformed with Agrobacterium strain LBA4404 harboring either PHP17215 or PHP17216 and produce embryogenic transformed calli as described (Zuo-yu Zhao et al. 2001, Molecular Breeding 8:323-333)

Culture in this manner was repeated as needed to obtain the amount of callus material to accomplish any analytical testing desired prior to regeneration of transformed plants. In the case of HI-IIMS45EX4, PCR-amplified products were hybridized with mutant- and wild-type-specific probes to determine copy number of the mutant ms45 gene and of the wild type Ms45 gene (endogenous plus transgene), to establish which lines were heterozygous and which were homozygous. The specific oligonucleotides used as PCR primers were TGCAGTACCCTCACCTCTTCTTC (SEQ ID NO: 1) and GCTTCACCGGCCGGTAGT (SEQ ID NO: 2). Probe oligonucleotides were TAGTCGCGGTGTCGCGGACC (mutant; SEQ ID NO: 3) and CCCTCATAGTCGCGGACCCG (wild type; SEQ ID NO: 4). Embryo-derived callus lines from male-sterile homozygous ms45/ms45 HI-IIMS45EX4 plants pollinated by male-fertile heterozygous Ms45/ms45 HI-IIMS45EX4 plants were typically about 50% homozygous ms45/ms45 and 50% heterozygous as expected. For male-fertile heterozygous Ms45/ms45 HI-IIMS45EX4 plants pollinated by male-fertile heterozygous Ms45/ms45 HI-IIMS45EX4 plants, about 25% homozygous ms45/ms45, 50% heterozygous Ms45/ms45 and 25% homozygous Ms45/Ms45 lines were typically obtained as expected.

Embryogenic stable callus were used for regeneration of transgenic plants and the transgenic plants were transferred to pots and grown in the greenhouse under standard conditions (Zuo-yu Zhao et al. 2001 supra).

Anthers of primary transgenic (T₀) plants, derived from HI-II transformed with Agrobacterium harboring PHP17215 or PHP17216 (see above), were stained for GUS activity (Jefferson 1987; McCabe, D. E., Swain, W. F., Martinell, B. J. and Christou, P. 1988. Stable transformation of soybean (Glycine max) by particle acceleration. Bio/Technology 6: 923-926.) at stages of development ranging from meiotic to mature (prior to extrusion and dehiscence). In addition, newly shed pollen was collected and cultured on pollen germination medium (Pfahler, P. L., Linskens, H. F. and Wilcox, M. 1980. In vitro germination and pollen tube growth of maize (Zea mays) pollen. IX. Pollen source genotype and nonionic surfactant interactions. Can. J. Bot. 58: 557-561) at 37° C. for 1-3 hr, then stained for GUS activity. Leaf pieces and root segments were stained for GUS activity as well. All GUS-stained plant materials were examined under a dissecting microscope to determine qualitatively the GUS activity. No GUS activity was observed in leaf pieces, root segments, anthers, meiocytes or microspores prior to Mitosis I. Starting at about Mitosis I, faint staining was observed in the microspores, becoming more pronounced in binucleate to mature pollen. Germinated pollen stained positive for GUS in both the pollen grain and the pollen tube. No difference was observed for plants transformed by Agrobacterium harboring either PHP17215 (˜2.8 Kb PG47 promoter fragment) or PHP17216 (˜1.2 Kb PG47 promoter fragment).

P67 and P95 promoters correspond to two maize genes, CPOAC67 and CPPAG95, respectively. (See US publication 20050246796). CPOAC67 and CPPAG95 are two maize pollen-expressed EST clones that showed limited homology to putative pectin methylesterase and putative L-ascorbate oxidase, respectively. The pollen specificity of these two clones has been confirmed by RT-PCR and Northern blot analyses of RNA samples from different tissues including leaf, root, anther/mature pollen grains, tassel at vacuole stage, spikelet, cob, husk, silk and embryo (see attached pictures). Southern blot analyses have shown that these clones represent single or low-copy genes in corn genome. Chromosome mapping using the oat chromosome substitution line revealed that CPOAC67 is located at Chromosome 1, and CPPAG95 is on Chromosome 6 and 8. These two EST clones have been used to screen a maize BAC library. Positive BAC clones have been found and subcloned into pBluescript KS. Subclones corresponding to the cDNA sequences have been identified and sequenced. The transcriptional start sites for these clones have been determined using a RNA ligase-mediated rapid amplification of 5′ end approach. The genomic sequence for CPOAC67 is 4074 bp in length, including a 1665 bp promoter region, an entire coding region and a 384 bp 3′ end sequence. The genomic sequence for CPPAG95 is 4035 bp, including a 1394 bp promoter region, an entire coding region and a 342 bp 3′ end sequence. Sequence comparisons between the genomic clones and EST cDNA clones revealed no intron for CPOAC67, two introns for CPPAG95.

### Example 2

### Analysis of PG47, P95 and P67 Promoters Using DAM and Alpha-Amylase Gene

The pollen specificity of these promoters was first examined using an E. coli DNA (Adenosine-N6) methyltransferase (DAM) gene which was previously shown to cause complete male sterility when expressed in anthers (E. Unger, S. Betz, R. Xu, A. M. Cigan, Transgenic Res, 10, 409-422, 2001). For PG47:DAM fusion gene (PHP18091), 47 transgenic corn plants were generated that are single copy and contain intact transgenes. Of these 47 plants, 23 plants (˜49%) were completely male sterile, 12 plants (24.5%) had a poor male fertility, and 12 plants (24.5%) showed near normal tassel phenotype. The complete male sterile phenotype is probably caused by non-pollen specific expression of the DAM gene. To test transgene transmission through pollen, pollen grains from poorly pollen-shedding plants were collected and used to pollinate non-transgenic plants. About 30 young embryos were harvested and plated on the medium containing bialaphos. All 30 embryos could not germinate, suggesting PG47::DAM fusion gene can block transgene transmission through pollen. Similar results were observed for P95::DAM and P67::DAM fusion genes. But most of the transgenic plants generated by P67::DAM showed normal or near normal male fertility. The phenotypical difference conferred by these three promoters reflects their difference in gene expression, such as timing and abundance. These three pollen promoters (PG47, P95 and P67) were then tested using a corn α-amylase gene. This α-amylase gene was isolated from a cDNA library made from developing kernels. Sequence analysis indicated that this α-amylase contains a putative signal peptide. Since starch accumulation in pollen occurs in amyloplasts, this putatative signal peptide was replaced by the amyloplast-targeting signal (BT1) from Brittle 1 gene (T. D. Sullivan, L. I. Strelow, C. A. Illingworth, R. L. Philips, O. E. Nelson, Jr., Plant Cell, 3, 1337-1348, 1991). All transformants generated by these three promoter and BT1::alpha amylase fusion gene showed normal phenotype including male fertility. But their ability in blocking transgene transmission through pollen is dramatically different from each other. For PG47::BT1::α-amylase fusion gene, 24 transgenic plants were generated and pollen grains from these plants were used to pollinate non-transgenic plants. About 100 young embryos from each cross were plated on herbicide medium. Embryos from 18 crosses did not germinate while embryos from 5 crosses showed ˜50% herbicide resistance and embryos from 1 cross show ˜30% herbicide resistance. Southern blot and PCR analyses revealed that these 5 T₀ plants contained no transgenic α-amylase and the T₀ plant showing 30% herbicide resistance contained multiple transgene insertions. This indicates that PG47::BT1::α-amylase fusion is able to prevent transgenic pollen from achieving fertilization. However, P95::BT1::α-amylase fusion showed ˜5% transgene escape rate via pollen and P67::BT1::α-amylase showed 50:50 transgene transmission rate through pollen. The result that P67::BT1::α-amylase showed no efficacy in terms of preventing transgene transmission through pollen suggests that alpha-amylase gene is not toxic or lethal to pollen. Thus, it is useful in an embodiment of the invention to screen promoters for “leakiness” and select those which have a highly preferred promoter expression.

### Example 3

### Alpha-Amylase Gene is Non-Lethal to Pollen But can Prevent Starch Accumulation

To understand the cytological changes of pollen grains that contain PG47::BT1::α-amylase fusion gene, we made a construct containing PG47::BT1::α-amylase linked to the PAT and dsRED (Dietrich et al. (2002) Biotechniques 2(2):286-293) fusion genes which are driven by the ubiquitin promoter (PG47::BT1::α-amylase-UBI::PAT::dsRED). Pollen grains at shedding stage but still within anthers were collected from transgenic plants and stained with FDA (fluorescein diacetate) for viability and with KI for starch accumulation. Most mature pollen grains showed normal pollen phenotype and fluorochrome reaction when stained with FDA, suggesting that they are still viable. When examined under fluorescence, about half of the mature pollen grains showed red fluorescent. These red fluorescent pollen grains did not show KI reaction while the non-fluorescent pollen grains were stained by KI-I₂. This indicates that transgenes can prevent starch accumulation in pollen. To examine the germination ability of the transgenic pollen, pollen grains at shedding stage were collected for in vivo germination tests. On the silks pollinated by these transgenic plants, about 70% of the red fluorescent pollen grains cannot re-hydrate and some red fluorescent pollen can. Very few red fluorescent pollen grains can germinate, but their pollen tube growth is very limited compared to control pollen.

To provide direct evidence that the alpha-amylase is not lethal to pollen, we made a construct containing PG47 promoter and alpha-amylase fusion gene without the Brittle1 transit peptide (PG47::α-amylase). When mature pollen from the transgenic plants containing PG47::α-amylase fusion gene were stained with KI-I₂, most pollen grains showed starch accumulation. This suggests that use of a targeting sequence to the amyloplast is preferred in situations where a starch degrading enzyme is used in a plant having starch highly expressed in amyloplast. Thus, the BT1 transit peptide is one of the amylose expression transit peptides preferred for use in directing starch accumulation in pollen. To verify that the alpha-amylase is indeed expressed in these transgenic pollen, pollen samples were used for Western blot analysis of gene expression. A clear band was detected in the samples, confirming that transgenic alpha-amylase is present in the pollen grains. To test whether the PG47::α-amylase without BT1 can prevent transgene transmission through pollen, pollen grains from transgenic plants were pollinated to non-transgenic plants. All transgenic plants showed 50:50 transgene transmission through pollen, suggesting alpha-amylase is not lethal to pollen.

### Example 4

### Recessive Male Sterile Mutants and Cloning of Male Fertility Genes

Three different male-sterile mutants, ms45, ms26 and ms22(msca1), have been tested in this invention. Both ms45 and ms26 mutants do not form a normal wall on the developing microspores which leads to microspore abortion soon after microspore release from tetrads. Ultrastructural studies show that there is little or no exine development in ms45 and ms26 mutants, possibly due to a defect in sporopollenin biosynthesis. The MS45 gene was isolated using an Activator transposon-tagging approach and was mapped to chromosome 9. A stable ms45 mutation was found from a perfect excision of Ac from the target gene, leaving the 8 base pair repeat which causes a frameshift mutation. MS45 showed limited homology to strictosidine synthase genes from Catharanthus roseus and Rauvolfia serpentina. The MS26 gene was isolated using a Mutator transposon-tagging approach and was mapped to chromosome 1. A stable ms26 mutant was also obtained from an excision allele that contains a frameshift mutation. The deduced MS26 protein appears to be a cytochrome P450 monooxygenase, similar to those from Arabidopsis thaliana and Vicia sativa that catalyze the omega-hydroxylation of fatty acids and alkanes. RNA gel blot analyses reveal that both MS45 and MS26 are anther-specific genes. The spatial expression of MS26 was further studied using in situ hybridization. It was found that MS26 is expressed specifically in tapetal cells within the anther. To demonstrate whether the cloned wild-type MS45 and MS26 genes can complement the male sterile phenotypes, both cDNA and genomic clones for MS45 and MS26, in combination with different anther-specific promoters (5126 and BS7), were transformed into ms45 and ms26 plants, respectively. It was found that a transformed copy of wild-type fertility gene was able to fully restore the male fertility phenotype to the male sterile mutants. The ms22 mutant does not undergo normal series of male gametogenesis and anthers in this mutant are transformed into vegetative organ-like structures. The MS22 gene was cloned through a map-based approach and showed similarity to plant glutaredoxin genes. Genetic complementation also demonstrated that the cloned copy of wild-type MS22 gene can restore male fertility to ms22 mutant.

### Example 5

### Test of Different Constructs for the Efficacy to Block Transgene Transmission Via Pollen

PG47::BT-1::α-amylase fusion (BT-1 referring to the brittle 1 nucleotide sequence) was further tested in combination with different adjacent promoters and genes, in different orientations. To plants generated from different constructs were subjected to Southern blot analysis for integration and copy number. Events that were single integration and contain intact transgenic polynucleotides of interests were then selected and evaluated for the transgene transmission via pollen. Pollen grains from these plants were used to pollinate non-transgenic inbred plants. Ears of 18 DAP old were harvested and examined for dsRED-expressed kernels under fluorescent microscope. The Table below summarizes the overall results for those constructs. (Reference to 35SENH refers to the single 35S enhancer, supra; AA refers to the alpha amylase sequence; 5126 is a promoter, supra.) The transmission rate varies with different constructs with the overall escape rate from these tests was ˜0.05%.

| | Transgene |
|---|---|
| | transmission rate |
| | through pollen(# of |
| | dsRED kernel/total |
| Constructs | kernel) |
| PHP20784(PG47::BT1::AA/5126::MS45/35S::Pat) | 0/1458 |
| PHP21478(PG47::BT1::AA/5126::MS45/UBI::moPat) | 0/700 |
| PHP24109(PG47::BT1::AA/MS45::MS45/LTP2::RFP) | 9/4155 (0.217%) |
| PHP24101(PG47::BT1::AA/MS26genomic/LTP2::RFP) | 2/3654 (0.055%) |
| PHP24418(PG47::BT1::AA/5126::MS45/LTP2::RFP) | 15/4927 (0.304%) |
| PHP24485(PG47::BT1::AA/5126:MS45/35SENH/LTP2::RFP) | 5/10922 (0.0458%) |
| PHP24490(PG47::BT1::AA/MS45P:Ms45/35SENH/LTP2:RFP) | 0/6456 |
| PHP24593(MS45::MS45/PG47::BT1::AA/35SENH/LTP2::RFP) | 0/1114 |
| PHP24612(MS45::MS45/PG47::BT1::AA(rev)/35SENH/LTP2::RFP) | 1/3784 (0.0264%) |
| PHP24597(5126::MS45/PG47::BT1::AA/35SENH/LTP2::RFP) | 3/3431 (0.0874%) |
| PHP24596(5126::MS45/PG47::BT1::AA(rev)/35SENH/LTP2::RFP) | 1/2531 (0.0395%) |

### Example 6

### Large Scale Test of Transgene Transmission Via Pollen

To test transgene transmission through pollen at a relatively large scale and to test the stability of transgenes over generations, transgenic plants from different constructs and at different generations were used as male parents and crossed to non-transgenic plants. Mature seeds harvested and examined for dsRED-expressed kernels. The results are shown as below. It was found the PG47::BT1::α-amylase fusion is stable over generations.

| | | | | | | | | T3 & T4* | | | | | | Total |
|---|---|---|---|---|---|---|---|---|---|---|---|---|---|---|
| | | T1 Transmission | | | T2 Transmission | | | Transmission | | | T5 Transmission | | | Transmission |
| Plasmid | Event | Red k | Yellow k | Rate | Red k | Yellow k | Rate | Red k | Yellow k | Rate | Red k | Yellow k | Rate | Rate (%) |
| PHP24597 | E6611.32.1.38 | 0 | 19012 | 0.000 | 2 | 86124 | 0.002 | 4 | 213685 | 0.002 | | | | 0.002 |
| PHP22625 | E6209.109.1.4 | 0 | 9354 | 0.000 | 3 | 16260 | 0.018 | 9 | 64120 | 0.014 | 6 | 213,748 | 0.003 | 0.006 |
| PHP24109 | E6499.75.6.3 | 4 | 9694 | 0.041 | 18 | 91490 | 0.020 | 61 | 143767 | 0.042 | | | | 0.034 |
| PHP24612 | E6611.22.8.2 | 1 | 11126 | 0.009 | 10 | 101264 | 0.010 | 71 | 146855 | 0.048 | | | | 0.032 |
| PHP24485 | E6499.105.1.5 | 2 | 7458 | 0.027 | 3 | 14,058 | 0.021 | 56 | 157329 | 0.036 | | | | 0.034 |
| PHP24597 | E6611.32.1.16 | ND | ND | ND | 10 | 52382 | 0.019 | 150 | 181932 | 0.082 | | | | 0.068 |
| PHP24485 | E6499.105.8.6 | 7 | 16755 | 0.042 | 4 | 58186 | 0.007 | 161 | 147018 | 0.110 | | | | 0.077 |
| PHP24485 | E6499.105.6.7 | 0 | 16624 | 0.000 | 20 | 103444 | 0.019 | 298 | 255143 | 0.117 | | | | 0.085 |
| PHP24596 | E6611.34.1.19 | 4 | 17964 | 0.022 | 24 | 98533 | 0.024 | 383 | 218481 | 0.175 | | | | 0.123 |
| PHP24485 | E6499.105.5 | 2 | 8291 | 0.024 | 19 | 104121 | 0.018 | 448 | 267199 | 0.168 | | | | 0.124 |

### Example 7

### Tests of Maintainer Fertility and the Maintenance of Transgenic Maintainer Plants

Transgenic plants containing PG47::BT1::alpha-amylase linked to cloned MS45 gene and dsRED gene and containing homozygous ms45/ms45 male sterile alleles were selfed. Progeny were tested for male fertility. In the table, the 5 events listed in this table are progeny plants derived from self pollination of hemizygous PG47::BT-1::alpha-amylase-MS45-dsRED transgenic plants. The first 5 rows showed the progeny with dsRED (and these plants are fertile plants since MS45 is present in these plants. The bottom 5 plants from the same events showed the progeny without dsRED and these plants should be male sterile plants since MS45 not present in these plants. These data confirmed that this transgene (PG47::BT-1::alpha-amylase-MS45-dsRED) can maintain the male fertility and it can transferred to progeny though female gametes since its pollen malfunctions. Also it confirmed that the male sterile status (ms45 mutants) can be maintained if MS45 fertile gene is not present in the plants.

### Corn SPT-Maintainer Fertility Testing

| | | | | | | | % Plants |
|---|---|---|---|---|---|---|---|
| | | | Fertile | Sectored | Shedder | Sterile | fully |
| Event | Loc | Type | Plants | Plants | Plants | Plants | Fertile |
| E6499.105.6.7 | HW | dsRED | 28 | 0 | 0 | 0 | 100% |
| E6611.32.1.38 | HW | dsRED | 188 | 6 | 0 | 0 | 96.91% |
| E6611.22.8.2 | HW | dsRED | 19 | 1 | 1 | 0 | 90.48% |
| E6499.75.6.3 | HW | dsRED | 15 | 2 | 0 | 0 | 86.67% |
| E6499.75.6.3 | JH | dsRED | 16 | 3 | 1 | 0 | 80.00% |
| E6499.105.6.7 | HW | Yellow | 0 | 0 | 0 | 14 | |
| E6611.32.1.38 | HW | Yellow | 0 | 0 | 0 | 16 | |
| E6611.32.1.38 | JH | Yellow | 0 | 0 | 0 | 19 | |
| E6611.22.8.2 | HW | Yellow | 0 | 0 | 0 | 13 | |
| E6499.75.6.3 | HW | Yellow | 0 | 0 | 0 | 16 | |
| E6499.75.6.3 | JH | Yellow | 0 | 0 | 0 | 15 | |

### Example 8

### Propagation of Recessive Genetic Male Sterile (ms) Plants

The propagation of ms plants was tested using a construct containing: PG47::BT1::AA/5126::MS45/35S::PAT When this construct was transformed into homozygous ms45 plants, all T₀ plants showed normal fertile tassel phenotype, suggesting that the wild-type MS45 clone in this construct can complement the male-sterile mutation and PG47::BT1::AA does not affect normal plant growth and development. To test the male sterility maintenance of this construct, pollen grains from transgenic plants were pollinated to non-transgenic homozygous ms45 plants. About 3,357 progenies from these crosses were grown and 3,356 plants showed ms45 male-sterile phenotype. This suggests that the transgenic plants can maintain and propagate homozygous ms45 male-sterile plants. One male-fertile plant that contained three transgenes was found. The transgene transmission rate through pollen in this test is about 0.03%.

### Example 9

### Inbred Conversion

Transgenic plants containing PG47::BT1::alpha-amylase linked to cloned MS45 gene and dsRED gene were converted into elite inbred lines in homozygous ms45 background. Pollen grains from these elite inbred lines pollinate to non-transgenic plants to test the transgene transmission via pollen at a relative large scale. The results were shown below. This indicates that PG47::BT1::alpha-amylase fusion gene can function across different genetic backgrounds.

| | | Transgene | | No. Red | Total No. Yel | | % Trans |
|---|---|---|---|---|---|---|---|
| Plasmid | Event | Background | # Ears | K's | K's | Trans Rate | Rate |
| PHP24597 | E6611.32.1.38 | PHETG | 229 | 0 | 78354 | 0.00000 | 0.000% |
| PHP24597 | E6611.32.1.38 | PH707 | 316 | 0 | 77108 | 0.00000 | 0.000% |
| PHP24597 | E6611.32.1.38 | PHC77 | 213 | 0 | 38421 | 0.00000 | 0.000% |
| PHP22625 | E6209.109.1.4 | PHEWW | 225 | 0 | 58972 | 0.00000 | 0.000% |
| PHP22625 | E6209.109.1.4 | PH707 | 312 | 6 | 71449 | 0.00008 | 0.008% |
| PHP22625 | E6209.109.1.4 | PHC77 | 190 | 7 | 13651 | 0.00051 | 0.051% |
| PHP24109 | E6499.75.6.3 | PHEWW | 209 | 12 | 55859 | 0.00021 | 0.021% |
| PHP24109 | E6499.75.6.3 | PH707 | 327 | 356 | 69303 | 0.00511 | 0.511% |
| PHP24109 | E6499.75.6.3 | PHC77 | 185 | 100 | 19195 | 0.00518 | 0.518% |
| PHP24485 | E6499.105.1.5 | PH51H | 215 | 10 | 57695 | 0.00017 | 0.017% |
| PHP24485 | E6499.105.1.5 | PH707 | 341 | 363 | 107254 | 0.00337 | 0.337% |
| PHP24485 | E6499.105.1.5 | PHC77 | 218 | 236 | 62016 | 0.00379 | 0.379% |

### Example 10

### Seed Sorting Test Using dsRED Gene

For seed-sorting tests, dsRED seeds and non-dsRED seeds were blended in proportions of 50:50 and 9995:5 mass that mimic the seeds for maintainer and female parent line, respectively. The seed blends were sorted using different settings. The seeds sorting was done using Satake Scanmaster high-speed color sorter. For 50:50 blends, the purity of dsRED seeds reached 88.1% after the first pass of sorting, and reached 98.0% after second pass of sorting. For 9995:5 blends, in most tests it only required two passes of sortings to reach 100% purity of non-dsRED seeds. The detailed seed-sorting results are shown as below. It is estimated that after 2 pass seed sorting (99.95% efficiency), the probability of the transgenic seeds is 1/285,700,000. In F₁ seeds sold to farmers (2× dilution of seed on the ear), the probability of the transgenic seeds is 1/571,400,000. In F2 grain harvested in farmer fields (2× dilution of seed on the ear), the probability of the transgenic seeds is 1/1,142,800,000 (8.7×10⁻¹⁰).

SIMS Purity Color Sorting
1:1 Maintainer:Sterile Mix
Lab: Kernels per Kg = 3293 (3293/2.2046 = 1493 k/lb)
Input Flow Rates:
Test: SM-II feed rate setting = 728
Calculated feed rate = 3418 lb/hr/bank (= 63.75 units/hr/bank)
Hedrick: Before sizing = 81 bu/hr/bank input @ 1.35% avg discard
Johnston: Before sizing = 67 bu/hr/bank @ 2.5% avg discard

| Pass # | Start (gm) | Accept (gm) | Initial % | Final Purity | % Steriles |
|---|---|---|---|---|---|
| 1 | 17,220 | 8,580 | 50% | 88.1% | 11.9% |
| 2 | 8226 | 8,228 | 96% | 98.0% | 2.0% |

### SPT Sterile Color Sorting Tests Low-Level dsRED Presence

Measured Single Pass Efficiency
5,322 Defects in 218 Pools 7,096 lb Total Weight 10,644,000 Total K

| | | Pass 1 | | Pass 2 | | Pass 3 | |
|---|---|---|---|---|---|---|---|
| Setup | N | Individual | Cumulative | Individual | Cumulative | Individual | Cumulative |
| 1 | 5 | 96.6667% | 96.6667% | 100.0000% | 100.0000% | 100.0000% | 100.0000% |
| 2 | 5 | 96.6667% | 96.6667% | 100.0000% | 100.0000% | 100.0000% | 100.0000% |
| 3 | 5 | 98.1667% | 98.1667% | 100.0000% | 100.0000% | 100.0000% | 100.0000% |
| 4 | 50 | 97.4722% | 97.4722% | 100.0000% | 99.9167% | 100.0000% | 100.0000% |
| 5 | 5 | 95.0000% | 95.0000% | 100.0000% | 100.0000% | 100.0000% | 100.0000% |
| 2 | 2 | 95.8333% | 95.8333% | 100.0000% | 100.0000% | 100.0000% | 100.0000% |

### Example 11

### Producing Herbicide and Insect Resistant Grass and Trees

The experiment is directed to producing grasses or trees that are herbicide and/or insect resistant or containing other traits that benefit for grass or tree growth. These grasses are either for harvesting its vegetative bodies (switch grass or pine trees) or for use of their vegetative bodies (turfgrass or pine trees), but not for seed harvesting. The key point is to block the pollen flow from these GMO grasses or threes to wild species or non-GMO grasses or trees. The methods are particularly useful in grasses and trees, including without limitation, for example, turfgrass, switch grass, bent grass, radiata pine and loblolly pine etc.

### Step-1:

Insert GAT/HRA/BT into MS22 locus though homologous recombination and make MS22 (dominant allele, male fertile) into ms22 (recessive allele, male sterile) and produce

$$hemizygous - \frac{GAT \text{/} HRA \text{/} BT \text{/} ms 22}{MS 22} .$$

Other genes, such as modification of the fibers, starch and other traits can also be used.

### Step-2:

$$Self this hemizygous - \frac{GAT \text{/} HRA \text{/} BT \text{/} ms 22}{MS 22}$$

and produce ¼ of the seeds containing

$$Homozygous - \frac{GAT \text{/} HRA \text{/} BT \text{/} ms 22}{GAT \text{/} HRA \text{/} BT \text{/} ms 22}$$

Hemizygous MS22/ms22 plants are 100% of pollen fertile, GAT/HRA/BT can be transmitted though pollen. Homozygous ms22/ms22, 100% pollen is sterile. Several methods can be used to separate these two different genotypes and seeds, such as QT-PCR, Southern blotting, or plant fertility check.

### Step-3:

Transform plants with RFP/PG47-BT-1-α-Amylase into MS22 background (but not into MS22 locus) and generate hemizygous—RFP/PG47-BT-1-α-Amylase/MS22 (50% pollen containing PG47-BT-1-α-Amylase and not functional).

### Step-4:

The above hemizygous—RFP/PG47-BT-1-α-Amylase is used as the female parent and use hemizygous—GAT/HRA/BT/ms22 as male parent (100% pollen fertile) to produce seeds. In these seeds, 25% of seeds are hemizygous at MS22 locus as—

$$\frac{RFP \text{/} PG47 \text{-} BT \text{-} 1 \text{-} α \text{-} Amylase \text{/} MS 22}{GAT \text{/} HRA \text{/} BT \text{/} ms 22}$$

Male gametes

| | | |
|---|---|---|
| Female | GAT/HRA/BT/ms22 | W.T. (MS22) |
| gametes | | |
| RFP/PG47-BT- | RFP/PG47-BT-1-α- | RFP/PG47-BT-1-α- |
| 1-α-Amylase/ | Amylase/MS22 | Amylase/MS22 |
| MS22 | GAT/HRA/BT/ms22 | |
| Wild-type | GAT/HRA/BT/ms22 | W.T. |
| (W.T.) (MS22) | | |

These ¼ of the seeds are different from the other ¾ of the seeds. These ¼ seeds are red color due to RFP and herbicide resistant due to GAT/HRA gene. Therefore these seeds and the plants derived from these seeds can be separated from other seeds and derived plants. These seeds and its derived plants are 100% pollen fertile and contain the trait genes and these seeds and its derived plants are used as the maintainer line for seed production.

All of these 4 steps are done in a controlled condition to avoid pollen flow.

### Step-5:

Use homozygous—GAT/HRA/BT/ms22 (male sterile) as the female line, and use hemizygous at MS22 locus—RFP/PG47-BT-1-α-Amylase/MS22/GAT/HRA/BT/ms22 as the male parent (˜50% pollen containing α-Amylase- not function, ˜50% pollen containing GAT/HRA/BT/ms22-functional pollen due to MS22 presence in their pollen mother cells) to produce seeds. Therefore, the pollen can transmit only GAT/HRA/BT/ms22 to the resulted seeds.

These seeds are 100% homozygous—GAT/HRA/BT/ms22, they grow normally and they contain all of the trait genes, such as GAT/HRA/BT or other trait genes to protect these plants or these plants can make special starch or fibers etc. But these plants are 100% male sterile, no viable pollen is produced from these plants. Therefore, there is no GMO pollen flow issue in these plants and these plants can not produce seeds that can fall on the ground easily to contaminate the environment.

These seeds are used commercially for golf courts and switch grass planting etc.

### Example 12

### Producing Herbicide and Insect Resistant Crops with α-Amylase Gene to Block GMO Pollen Flow

This experiment uses sorghum as an example to describe how the GMO pollen is blocked in the transgenic crops. A number of crop species can cross-pollination with some wild species, such as sorghum vs. johnson grass, maize vs. teosinte. This experiment describes the physical linkage of all transgenes (including input trait genes including, but not limiting to insect resistant genes and herbicide tolerant genes and output trait genes including, but not limiting to drought tolerance genes, cold tolerance genes, high lysine genes etc. inserted into plant genome) to the pollen disruption component and making the pollen containing any of these transgenes malfunction for pollination.

### Step-1:

Insert a construct into the genome of sorghum variety TX430, P898012 or Macia or other sorghum varieties, such as ICSV112, Seredo and ICSV111 1N etc. through genetic transformation. These constructs include this pollination-disruption component directly linked to trait genes, such as an insect resistant gene (Bt) and herbicide resistant genes (glufosinate resistance, GAT and HRA) or other trait genes, such as drought tolerance, nitrogen utilization, cold tolerance genes. An example of these constructs is PG47::BT-1::α-Amylase::Bt::GAT::HRA::GZ promoter::phytoene synthase::phytoene desaturase::lycopene β-cyclase. This construct also includes a β-carotene or provitamin-A genes (phytoene synthase::phytoene desaturase::lycopene β-cyclase) providing golden color for the seeds as a color marker. Through transformation, transgenic T₀ sorghum plants are produced. These T₀ plants are hemizygotes for this inserted construct.

Other seed-specific promoters such as FL-2, CZ19B1, OLE, EAP1, etc. can be used to drive β-carotene or provitamin-A genes.

### Step-2:

Plant T₀ plants and produce the seeds (T₁ seeds) which contain this construct. The herbicide and insect resistant genes (or other trait genes) are beneficial for the plant growth. These T₀ plants produce two kinds of pollen: 50% pollen containing this construct and they are malfunctional (no GMO pollen flow) due to α-amylase gene; and 50% pollen not containing this construct and they are non-GMO with viable pollen. This non-GMO pollen is the only functional pollen source for pollination. These T₀ plants produce two kinds of female gametes, 50% of the female gametes contining this construct and 50% of the female gametes not containing this construct and both kinds of the female gametes are functional normally. The seeds produced from these T₀ plants are 50% of the seeds containing hemizygotes of this construct (GMO seeds) and 50% of the seeds not containing this construct (non-GMO seeds).

| | Pollen | |
|---|---|---|
| | GMO: α-amylase & β- | |
| Female gametes | carotene | Non-GMO |
| GMO: α-amylase | Female gamete: viable | Female gamete: viable |
| & β-carotene | Pollen: pollination-disruption | Pollen: viable |
| | No pollination made & no | Produce GMO seeds |
| | seed produced | (50%) |
| Non-GMO | Female gamete: viable | Female gamete: viable |
| | Pollen: pollination-disruption | Pollen: viable |
| | No pollination made & no | Produce non-GMO |
| | seed produced | seed (50%) |

### Step-3:

Separate the GMO and non-GMO seeds mechanically by color of the seeds. Since β-carotene (or provitamin A) containing seeds give golden color to the seeds, such as golden rice. The seeds containing this construct (GMO seeds) are golden colored and the seeds not containing this construct (non-GMO seeds) are regular color. The GMO and non-GMO seeds can be sorted by a machine as described in Example 9.

### Step-4:

The golden color seeds (GMO) are used for commercialization and field planting and the regular color seeds (non-GMO) are used for food and feed and/or other industry materials or for field planting of non-GMO crops.

When these GMO seeds (hemizygote for this construct) are planted and the derived plants contain this construct—herbicide and insect resistant genes (or other trait genes). These genes are benefit for the plant growth. Same as the T0 plants, the pollen produced from these GMO plants are: 50% of pollen containing this construct and they are pollination-disruption (no GMO pollen flow) and 50% of the pollen not-containing this construct and they are normal viable non-GMO pollen. This normal, viable, and non-GMO pollen is the only source of functional pollen to pollinate these plants to produce seeds (grains).

These seeds harvested from these plants will be sorted by a machine as described in Step-3 to separated GMO and non-GMO seeds for different applications.

### Example 13

### Producing Herbicide and Insect Resistant Hybrids with α-Amylase Gene to Block GMO Pollen Flow

This experiment describes the method of making transgenic (GMO) hybrids in crops. In these hybrids, the GMO-pollen is disrupted for pollination.

### Step-1:

Insert a construct into the genome of a male sterile (MS) sorghum line such as 296A through genetic transformation. These constructs include, but not limit to this construct: PG47::BT-1::α-Amylase::Bt::GAT::HRA::GZ promoter::phytoene synthase::phytoene desaturase::lycopene β-cyclase. Through transformation, transgenic T₀ plants are produced. These T₀ plants are hemizygotes for this inserted construct.

Other seed-specific promoters such as FL-2, CZ19B1, OLE, EAP1, etc. can be used to drive β-carotene or provitamin-A genes.

### Step-2:

These T₀ plants are male sterile (no GMO pollen flow) due to its male sterile (MS) nature. Pollinate these T₀ plants (as female) with pollen from a non-transformed maintainer sorghum line such as 296B for transgenic 296A (MS) seed increase. Due to the T₀ plants being hemizygotes for this construct and the non-transgenic 296B, the seeds are 50% containing hemizygotes of this construct (GMO seeds) and 50% not containing this construct (non-GMO seeds).

| Female gametes | |
|---|---|
| (from transgenic 296A) | Non-GMO Pollen from 296B |
| GMO: α-Amylase & β-carotene (or | Female gamete: viable |
| provitamin A) | Pollen: viable |
| | Produce GMO seed, golden color |
| | (50%) |
| Non-GMO | Female gamete: viable |
| | Pollen: viable |
| | Produce non-GMO seed, regular |
| | color (50%) |

Most male sterile (MS) sorghum lines are not 100% male sterile. They still produce a small amount of pollen. In this case, pollen produced from this transgenic 296A is either GMO-pollen or non-GMO pollen. However, all GMO-pollen contains the α-amylase gene that leads to pollination-disruption. Therefore, even though 296A is not 100% male sterile, there is still no functional GMO-pollen flowing from these transgenic 296A plants.

### Step-3:

Separate the GMO and non-GMO seeds mechanically. B-carotene or provitamin A-containing seeds can give a golden color to the seeds, such as golden rice. The seeds containing this construct (GMO seeds) are golden colored and the seeds not containing this construct (non-GMO seeds) are regular color. The GMO and non-GMO seeds can be sorted by a machine as described in Example 9. The regular color seeds (non-GMO) are used for food and feed and/or other industry materials or used as the female parent to make non-GMO hybrids. The golden color (GMO) seeds are used as the female parent to make GMO hybrids.

### Step-4:

The golden color seeds (GMO seeds) are planted and the resulting plants are used as the female parent for the hybrid. This female parent is male sterile (MS) GMO plant and they do not produce functional pollen (no GMO pollen flow). There is no de-tasselling or removal of anthers needed. These female plants are pollinated with a non-GMO restorer sorghum line (most sorghum lines are restorer lines) as the male parent to make hybrid seeds.

### Step-5:

These hybrid seeds from the above cross are 50% golden color (GMO) seeds and 50% regular color (non-GMO) seeds since the female parent (MS-GMO) is hemizygotic for the construct. These two kinds of seeds are sorted mechanically. The golden color seeds are the GMO hybrid seeds for commercialization and field planting. The regular color seeds (non-GMO) also can be used as commercial non-GMO hybrid seeds and field planting.

### Step-6:

The GMO hybrid seeds (hemizygotic for the construct) are planted in the field and the derived hybrid plants contain this construct, herbicide and insect resistant genes (or other trait genes). These genes are beneficial for the plant growth. These hybrid plants restore fertility and produce pollen. However, the pollen produced from these GMO hybrid plants are: 50% of pollen containing this construct and they are pollination-disruption (no GMO pollen flow) and 50% of the pollen not-containing this construct and they are normal viable non-GMO pollen. This normal viable non-GMO pollen is the only functional pollen source to pollinate these plants for grain production.

This diagram summarizes the above 6 Steps.

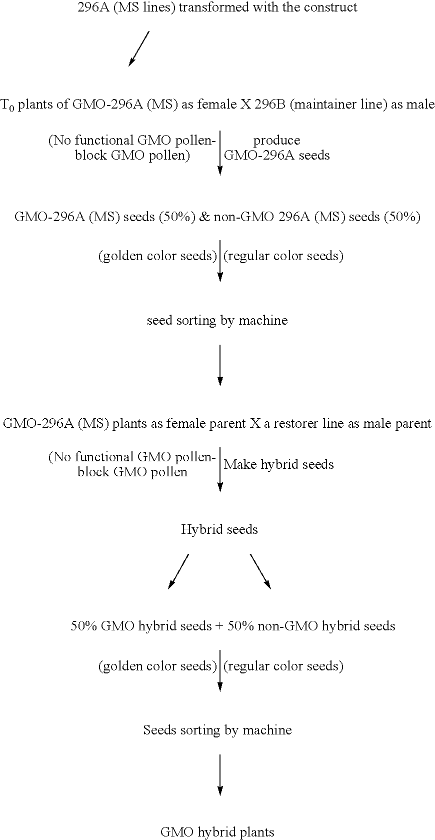

(GMO hybrid plants produce 50% GMO pollen containing α-amylase leading to malfunctional pollen and 50% non-GMO pollen that are viable for pollination—block GMO pollen)

### Example 14

### Producing Herbicide and Insect Resistant Hybrids with α-Amylase Gene and Molecular Recombination Systems to Block GMO Pollen Flow

This experiment describes another method of generating transgenic hybrids with the pollen disruption component by using molecular recombination systems. Sorghum is used as an example crop here. This method can be used in any plant species.

### Step-1:

Make two constructs for genetic transformation.

### Construct-1: lox: UBI::FLP::LTP2::YFP::lox

Construct-2: PG47::BT-1::frt1::UBI::CRE::LTP2::RFP::frt1::α-amylase::Bt::GAT::HRA
LTP2 is a seed-specific promoter from a gene coding Lipid Transfer Protein in barley. (See Opsahl-Sorteberg et al. “Identification of a 49-bp fragment of the HvLTP2 promoter directing aleurone cell specific expression” Gene 341 (2004) 49-58).
YFP is yellow fluorescent protein; it is used as a visible marker to indicate the existing or not existing of the DNA fragment of lox::UBI::FLP::LTP2::YFP::lox in the seeds
RFP is red fluorescent protein; it is used as a visible marker to indicate the existing or not existing of the DNA fragment of frt1::UBI::CRE::LTP2::RFP::frt1 in the seeds.
In construct-2, promoter PG47::BT-1 is separated with α-amylase gene by the sequence of frt1::UBI::CRE::LTP2::RFP::frt1. The α-amylase gene does not function in this construct. The pollen produced from this transgenic plant is fertile.

### Step-2:

Make sorghum line 296B (maintainer line) homozygous for the construct-1 through genetic transformation and self pollination.
Transform construct-1 (lox::UBI::FLP::LPT2::YFP::lox) into genome of sorghum line 296B and regenerate T₀ plants.
Self these T₀ plants to produce T₁ seeds. The T₁ seeds have two different colors under UV light, yellow fluorescent and non-yellow fluorescent. These yellow fluorescent seeds are transgenic seeds containing construct-1 and the non-yellow fluorescent seeds are non-transgenic seeds. Plant the transgenic T₁ seeds and grow into transgenic T₁ plants. Analyze these T₁ plants with quantitative PCR to determine which plants are homozygous for the construct-1 DNA fragment. Self these homozygous transgenic plants and obtain homozygous transgenic T₂ seeds.

296B is fully fertile plants and all pollen produced from these T₀ plants are viable. To avoid pollen flow, growth and pollination process of this plant should be completed in a contained greenhouse.

### Step-3:

Make sorghum line 296A (male sterile line) homozygous for construct-1 through cross between 296A and homozygous transgenic 296B obtained in Step-2.
Use 296A as female and pollinate these plants with pollen from 296B homozygous transgenic plants obtained in Step-2 to produce seeds of transgenic 296A. These transgenic 296A seeds are 100% hemizygous transgenic seeds containing construct-1. All of the seeds are yellow fluorescent under UV light.
Use these hemizygous transgenic 296A as female plants and pollinate them with pollen from 296B homozygous transgenic plants obtained in Step-2 again to produce seeds of transgenic 296A. These transgenic 296A seeds are 50% homozygous and 50% hemizygous for construct-1. Both are yellow fluorescent under UV light.
Select 296A homozygous-transgenic plants by quantitative PCR analysis and pollinate these plants by pollen from 296B homozygous transgenic plants to maintain 296A homozygous transgenic status. All of the 296A seeds are 100% homozygous for construct-1.

These processes are also handed in a contained greenhouse for the same reasons mentioned in Step-2.

### Step-4:

Make sorghum line 296B (maintainer line) homozygous for the construct-2 through genetic transformation and self pollination.
ITransform construct-2 (PG47::BT-1::frt1::UBI::CRE::LTP2::RFP::frt1::α-Amylase::Bt::GAT::HRA) into genome of sorghum line 296B and regenerate T₀ plants.
Self these T₀ plants to produce T₁ seeds. The T₁ seeds have two different colors under UV light, red fluorescent and non-red fluorescent. These red fluorescent seeds are transgenic seeds containing construct-2 and the non-red fluorescent seeds are non-transgenic seeds. Plant the transgenic T₁ seeds and grow into transgenic T₁ plants. Analyze these T₁ plants with quantitative PCR to determine which plants are homozygous for the construct-2 DNA fragment. Self these homozygous transgenic plants and obtain homozygous transgenic T₂ seeds.
Promoter PG47::BT-1 and α-amylase are disconnected by the sequence of frt1::UBI::CRE::LTP2::RFP::frt1. The α-amylase gene does not function in this construct. The pollen produced from this transgenic plant is fertile. These processes should be completed in a contained greenhouse.

### Step-5:

Make sorghum line 296A homozygous for PG47::BT-1::frt1::α-amylase::Bt::GAT::HRA
Use homozygous 296A with construct-1 (lox::UBI::FLP::LTP2::YFP::lox) obtained in Step-3 as female to cross by homozygous 296B with construct-2 (GP47::BT1::frt1::UBI::CRE::LPT2::RFP::frt1::α-amylase::BT::GAT::HRA) obtained in Step-4 as male. In this cross, all of the female gametes contain the DNA insert of construct-1 (lox::UBI::FLP::LTP2::YFP::lox) and all of the male gametes contain the DNA insert of construct-2 (GP47::BT-1::frt1::UBI::CRE::LPT2::RFP::frt1::α-amylase::Bt::GAT::HRA). When the male gametes fertilize the female gametes to form zygotes or after the zygotes formed, the recombinases encoded either by FLP and by CRE respectively are active for molecular recombination occurred at frt and lox sites. These two of simultaneous recombinations result in the formation of a new DNA fragment in the cells: lox::GP47::BT-1::frt1::α-amylase::Bt::GAT::HRA to generate homozygous transgenic 296A with this newly formed DNA insert. In this newly formed DNA insert, GP47::BT-1 is directly linked to the α-amylase gene and this linkage gives rise to malfunctional pollen (pollination-disruption) that contains Bt+GAT+HRA genes or other trait genes. In this newly formed DNA insert, the frt site between the GP47 promoter and the α-amylase gene is in coding frame or in an intron; it does not disrupt the function of GP47 promoter and the coding region of α-amylase gene. In the newly formed DNA insert, both YFP and RFP are excised from the genome. Therefore, the resulted seeds should not contain YFP and RFP genes and these seeds are not yellow and/or Red fluorescent under UV light. Any seeds with either yellow and/or red fluorescent color under UV light should be discarded; they may come from the incomplete recombination.
These seeds are 296A with homozygous insert of lox::GP47::BT-1::frt1::α-amylase::Bt::GAT::HRA. These seeds are used to make transgenic hybrid seeds.

These processes are also handed in a contained greenhouse for the same reasons mentioned in Step-4.

### Step-6:

Make transgenic hybrid seeds.
Use this 296A homozygous transgenics with the insert of lox::GP47::BT-1::frt1::α-amylase::Bt::GAT::HRA as female parent and use non-transformed sorghum restorer line as male parent to make cross. Since this GMO-296A is a MS line and it also contains GP47::BT-1::α-amylase gene, all of its pollen are malfunctional. The only viable source of pollen is from the male parent: the non-GMO restorer line.
Through this cross, 100% of the F₁ hybrid seeds are GMO seeds. There is no need to sort the GMO and non-GMO seeds as in Example 11 and 12.

Since there is no GMO pollen flow issue, this process can be done in the field.

### Step-7:

Grow the hybrid in the field.
All the hybrid seeds obtained in Step-6 are hemizygous for lox::PG47::BT-1::frt1::α-amylase::Bt::GAT::HRA and these plants are protected from Bt::GAT::HRA genes (or other trait genes).
Pollen produced from these plants is 50% GMO pollen containing lox::PG47::BT-1::frt1::α-amylase::Bt::GAT::HRA and 50% non-GMO pollen. All pollen containing lox::PG47::BT-1::frt1::α-amylase::Bt::GAT::HRA are malfunctional. This non-GMO pollen is the only pollen source for pollination. Therefore, there is no GMO pollen flow issue.

This diagram summarizes the above 7 Steps.

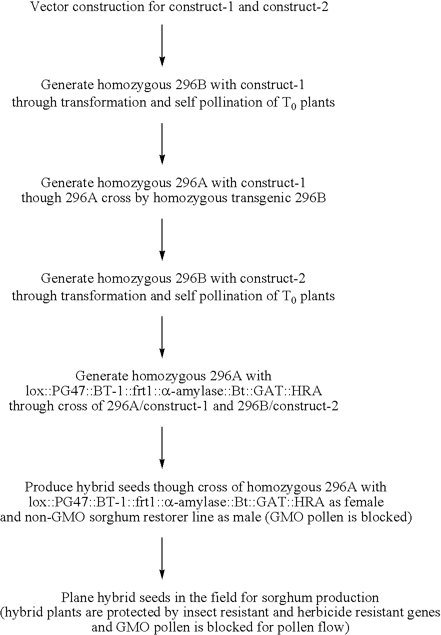

As it can be seen, the invention achieves at least all of its objectives.

What is claimed is:

1. A recombinant nucleotide construct for the purpose of blocking the transmission of a transgenic polynucleotide of interest into other sexually compatible plants comprising a promoter directing expression to pollen, operably linked to a pollination-disruption polynucleotide that renders the pollen unable to fertilize a sexually compatible ovule, wherein the promoter and the pollination-disruption polynucleotide are linked to at least one transgenic polynucleotide of interest.

2. The recombinant nucleotide construct of claim 1, wherein the pollination-disruption polynucleotide is not cytotoxic to pollen.

3. The recombinant nucleotide construct of claim 1, wherein the pollination-disruption polynucleotide is not cytotoxic to a plant cell.

4. The recombinant nucleotide construct of claim 1, wherein the pollination-disruption polynucleotide disrupts the storage source of the pollen so that the pollen is not capable of re-hydrating, germinating, producing a pollen tube, or releasing sperm cells.

5. The recombinant nucleotide construct of claim 1, wherein the construct further comprises a leader sequence operably linked to the pollination-disruption polynucleotide, wherein the leader sequence directs the peptide encoded by the pollination-disruption polynucleotide into a pollen organelle.

6. The recombinant nucleotide construct of claim 5, wherein the leader sequence is selected from the group consisting of a brittle 1, prSS, prCAB, and plastid-imported nucleic acid molecule.

7. The recombinant nucleotide construct of claim 5, wherein the pollen organelle is an amyloplastid, mitochondria, protein body, oil body or other compartments in pollen for storing energy sources or enzymes that are critical for pollen re-hydration, germination, pollen tube growth, sperm cell release, and fertilization.

8. The recombinant nucleotide construct of claim 1 wherein the promoter is selected from the group consisting of a PG47, P67, P95 and 5126 promoter.

9. The recombinant nucleotide construct of claim 1 wherein the pollination-disruption polynucleotide disrupts starch accumulation in pollen.

10. The recombinant nucleotide construct of claim 1 wherein the pollination-disruption polynucleotide is an alpha-amylase, beta-amylase, a starch debranching enzyme, Sugary1, pullulanase, glucanase, and SacB gene.

11. The recombinant nucleotide construct of claim 1 wherein the transgenic polynucleotide of interest is selected from the group consisting of a polynucleotide impacting insecticide resistance, disease resistance, herbicide resistance, drought tolerance, cold tolerance, nitrogen utilization, nutrition content, cellulose content, male sterility, female sterility, abiotic stress resistance, antibiotic resistance, site specific DNA integration, and selection of a linked nucleotide.

12. The recombinant nucleotide construct of claim 11 wherein the transgenic polynucleotide of interest is a polynucleotide that impacts herbicide resistance is selected from the group consisting of a polynucleotide providing resistance to glyphosphate, glyfosinate, a sulfonylurea herbicide, an imidazoline herbicide, a hyrdoxyphenylpyruvate dioxygenase inhibitor, and a protoporphyrinogen oxidase inhibitor.

13. The recombinant nucleotide construct of claim 1 wherein the transgenic polynucleotide of interest is a polynucleotide encodes Bacillus thuringienesis.

14. The recombinant nucleotide construct of claim 1 wherein the transgenic polynucleotide of interest is a gene that confers resistance to striga.

15. The recombinant nucleotide construct of claim 1 wherein the transgenic polynucleotide of interest is a male fertility gene selected from the group consisting of MS45, MS26 and MS22.

16. The recombinant nucleotide construct of claim 1 wherein the construct further comprises a non lethal marker.

17. The construct of claim 16, wherein the non lethal marker is selected from the group consisting of a polynucleotide encoding beta carotene, a fluorescent protein, and anthocyanin.

18. The construct of claim 16, wherein the non lethal marker encodes a beta carotene.

19. The recombinant nucleotide construct of claim 1 wherein the construct comprises a second promoter that drives the expression of the transgenic polynucleotide of interest

20. The recombinant nucleotide construct of claim 1 wherein the second promoter is selected from the group consisting of ubiquitin, lipid transfer protein from barley, or CaMV 35 LTP2, END2.

21. A plant comprising in its genome the recombinant nucleotide construct of claim 1.

22. The plant of claim 21, wherein said plant is an angiosperm or gymnosperm.

23. The plant of claim 21, wherein the plant is corn.

24. The plant of claim 21, wherein the plant is sorghum.

25. Seeds obtained from the plant of claim 21.

26. A plant, yeast or bacterial cell comprising the recombinant nucleotide construct of claim 1.

27. Pollen or ovule of the plant of claim 21.

28. The plant of claim 21 wherein the plant is a grass or pine tree.

29. A vector comprising the recombinant nucleotide construct of claim 1.

30. A method for producing a plant that will not produce functional transgenic pollen comprising:
transforming a plant cell with a recombinant nucleotide construct comprising a promoter directing expression to pollen, operably linked to a pollination-disruption polynucleotide that renders the pollen malfunctional, wherein the combination of the promoter and the pollination-disruption polynucleotide are linked to at least one transgenic polynucleotide of interest; and
regenerating a plant from the transformed plant cell.

31. The method of claim 30 wherein the generated plant is hemizygous with respect to the recombinant nucleotide construct.

32. The method of claim 30 further comprising selfing the generated plant to produce progeny maintaining the hemizygosity of the plant.

33. The method of claim 30 further comprising vegetatively propagating the generated plant to produce progeny.

34. A transgenic plant produced by the method of claim 30.

35. Seeds obtained from the plant produced by the method of claim 30.

36. A method for producing a transgenic seed that when grown into a plant produces malfunctional pollen comprising:
producing a first plant that contains in its genome a recombinant nucleotide construct comprising a promoter directing expression to pollen, operably linked to a pollination-disruption polynucleotide that renders the pollen malfunctional, operably linked to a marker,
crossing the first plant with a second plant;
harvesting the resultant seed, and
identifying seed comprising the construct such that the seed when grown into a plant produces malfunctional pollen.

37. The method of claim 36, wherein the marker is a non-lethal marker, and the seed comprising the construct is identified by identifying seed having the marker.

38. The method of claim 36, wherein the marker is operably linked to a promoter directing expression to seed tissue of the plant.

39. The method of claim 37, wherein the marker encodes a beta carotene protein.

40. The method of claim 36, wherein the marker is a selectable marker and seed comprising the construct is identified by exposing seed to the selection agent.

41. The method of claim 40, wherein the selectable marker is a herbicide resistance marker.

42. The method of claim 36, further comprising producing plants from seed identified having the construct, and crossing the resulting plant with a third plant not having the construct to produce second seed, such that the second seed having the construct produce plants with malfunctional pollen.

43. The method of claim 36, wherein the first or second parent plant is an inbred, hybrid plant or cultivar.

44. The method of claim 36, wherein the first parent plant is a male-sterile female plant.

46. A method of producing seeds comprising:
(i) transforming a male sterile plant with a construct comprising at least one nucleic acid molecule encoding a first recognition site and a first enzyme which recognizes a second recognitions site, a promoter directing expression to seed tissue operably linked to a first marker;
(ii) transforming a second male fertile plant with a construct comprising at least one nucleic acid molecule encoding a second recognition site and a second enzyme recognizing the first recognition site, a promoter directing expression to seed tissue operably linked to a pollination disruption polynucleotide and a second marker;
(iii) crossing the male sterile plant with the male fertile plant such that the second enzyme cleaves the first recognition site and the first enzyme cleaves the second recognition site, thereby activating the pollination disruption polynucleotide;
(iv) producing seed from the cross having the construct and that when grown into a plant produces malfunctional pollen.

47. The method of claim 46, wherein the first marker and the second marker are different markers.

48. The method of claim 46 wherein the markers are non-lethal markers and further comprising identifying seed in which the first and second recognition sites are cleaved by identifying seed which does not have either first or second marker.

49. The method of claim 48, wherein the marker is selected from the group consisting of a polynucleotide encoding a beta carotene, a fluorescent protein, and anthocyanin.

50. The construct of claim 49, wherein the marker encodes a beta carotene.

51. The method of 46, further comprising providing a transgenic polynucleotide of interest in the second construct, producing plants from the seed identified has not having the first or second marker, and crossing the identified seed with a third plant not having a construct to produce plants having the gene of interest and which produce malfunctional pollen.

52. The method of claim 51, wherein the transgenic polynucleotide of interest is a polynucleotide that impacts herbicide resistance is selected from the group consisting of a polynucleotide providing resistance to glyphosphate, glyfosinate, a sulfonylurea herbicide, an imidazoline herbicide, a hyrdoxyphenylpyruvate dioxygenase inhibitor, and a protoporphyrinogen oxidase inhibitor.

53. The method of claim 52, wherein the transgenic polynucleotide of interest is a polynucleotide encodes Bacillus thuringienesis

54. The method of 46 wherein the first recognition site is a Lox recognition site and the second enzyme is a CRE enzyme.

55. The method of 46 wherein the second recognition site is a Lox recognition site and wherein the first enzyme is a CRE enzyme.

56. The method of 46 wherein the second recognition site is a FRT recognition site and wherein the first enzyme is a FLP enzyme.

57. The method of 46 wherein the first recognition site is a FRT recognition site and the second enzyme is a FLP enzyme.

58. The method of 46 wherein the plant is a sorghum plant.

59. The method of 46 wherein the recombinant nucleotide construct further comprises a leader sequence operably linked to the pollination-disruption polynucleotide, wherein the leader sequence directs the peptide encoded by the pollination-disruption polynucleotide into the pollen.

60. The method of 59 wherein the leader sequence is selected from the group consisting of a brittle 1, prSS, prCAB, and plastid-imported nucleic acid molecule.

61. The method of 46 wherein the promoter of the recombinant nucleotide construct is a selected from the group consisting of a PG47, P67, P95 and 5126 promoter.

62. The method of 46 wherein the pollination-disruption polynucleotide of the recombinant nucleotide construct is selected from the group consisting of an alpha-amylase, beta-amylase, a starch debranching enzyme, Sugary1, pullulanase, glucanase, or SacB gene.

63. The method of 46 wherein at least one of the markers is a herbicide or antibiotic resistance nucleic acid molecule.

64. A plant containing pollination-disruption polynucleotide that when expressed makes the pollen malfunctional produced by the method of claim 46.

65. Seeds obtained from the plant of claim 64.

66. A method for producing homozygous male-sterile plants for hybrid seed production comprising:
(a) crossing a sexually compatible male-sterile parent plant that is homozygous for a male-sterility with a maintainer line, wherein the maintainer line is homozygous for male-sterility in its genome and hemizygous in its genome for a recombinant nucleotide construct comprising a promoter directing expression to pollen, operably linked to a pollination-disruption polynucleotide that renders the pollen malfunctional, wherein the combination of the pollen-specific promoter and the pollination-disruption polynucleotide are linked to a selectable marker and a wild type male fertility gene that complements the male sterile mutation of the male parent; and
(b) harvesting the progeny seed of the cross of (a).

67. The method of claim 67, wherein the male-sterility of the male-sterile parent plant is not transgenic.

68. The method of claim 67, wherein the progeny seed of the cross of (a) produce non-transgenic hybrid seeds.

69. The method of claim 67, wherein the method further comprises producing homozygous male-sterile plants by growing the non-transgenic seeds that contain the male sterility gene.

70. A hybrid plant obtained by the method of claim 66.

71. A male-fertile parent plant for maintaining a male-sterile line of a plant comprising a male-sterile parent plant that is homozygous for male-sterility in its genome and hemizygous in its genome for a recombinant nucleotide construct comprising a pollen-specific promoter operably linked to a pollination-disruption polynucleotide that renders the pollen malfunctional, wherein the combination of the pollen-specific promoter and the pollination-disruption polynucleotide are linked to a selectable marker and a wild type male fertility gene that complements the male sterile mutation of the male parent.

72. A method for establishing a population of transgenic plants containing the recombinant nucleotide construct of claim 1, the method comprising producing a plant that is hemizygous for the construct of claim 1, selfing the plant to produce progeny, and selecting from the progeny transgenic plants having the construct.

73. A method of selecting plant seed having a nucleotide sequence comprising operably linking with the nucleotide sequence a second nucleotide sequence encoding a beta carotene and identifying seed having the second nucleotide sequence.

74. The method of claim 73, wherein the second nucleotide sequence is operably linked to a promoter directing expression to seed tissue.

75. The method of claim 73, wherein seed having the second nucleotide sequence are sorted from seed not having the second nucleotide sequence by sorting seed by color.


SEQUENCE LISTING (4 sequences)

SEQ ID NO: 1 - DNA, 23 bp, Artificial Sequence
  Description of Artificial Sequence Synthetic primer
tgcagtaccctcacctcttcttc

SEQ ID NO: 2 - DNA, 18 bp, Artificial Sequence
  Description of Artificial Sequence Synthetic primer
gcttcaccggccggtagt

SEQ ID NO: 3 - DNA, 20 bp, Artificial Sequence
  Description of Artificial Sequence Synthetic probe
tagtcgcggtgtcgcggacc

SEQ ID NO: 4 - DNA, 20 bp, Artificial Sequence
  Description of Artificial Sequence Synthetic probe
ccctcatagtcgcggacccg